(19) United States
(12) Patent Application Publication
(10) Pub. No.: US 2023/0132907 A1
(43) Pub. Date: May 4, 2023

(54) CARDIAC VALVE REPAIR DEVICES, AND ASSOCIATED METHODS AND SYSTEMS

(71) Applicant: Half Moon Medical, Inc., Menlo Park, CA (US)

(72) Inventors: Matthew McLean, San Francisco, CA (US); Jose Gonzalez, Fremont, CA (US); Guarav Krishnamurthy, Mountain View, CA (US); Neil Zimmerman, Menlo Park, CA (US); Robert O'Grady, San Francisco, CA (US); Hanson S. Gifford, III, Woodside, CA (US)

(21) Appl. No.: 17/981,221

(22) Filed: Nov. 4, 2022

Related U.S. Application Data
(60) Provisional application No. 63/275,907, filed on Nov. 4, 2021.

(51) Int. Cl. A61F 2/24

(52) U.S. Cl. CPC A61F 2/2445; A61F 2230/0013; A61F 2220/0041; A61F 2220/0075; A61F 2230/0017

(57) ABSTRACT

Cardiac valve repair devices and associated systems and methods are disclosed herein. A cardiac valve repair device configured in accordance with embodiments of the present technology can include, for example, a coaptation member and an atrial-fixation member. The coaptation member can comprise an inner portion having a coaptation surface configured to coapt with a first native leaflet during systole and an outer portion configured to displace at least a portion of a second native leaflet. The atrial-fixation member can comprise a plurality of interconnected struts having a circumferential U-like shape about a flow axis of the cardiac valve and defining a brim portion and a pair of connection portions. The connection portions can be coupled to the coaptation member, and the brim portion can be configured to press against cardiac tissue above the first native leaflet proximate to a native valve annulus of the cardiac valve.

### CROSS-REFERENCE TO RELATED APPLICATION

This application claims the benefit of U.S. Provisional Patent Application No. 63/275,907, filed Nov. 4, 2021, and titled “CARDIAC VALVE DEVICES, AND ASSOCIATED METHODS AND SYSTEMS,” which is incorporated herein by reference in its entirety.

### TECHNICAL FIELD

The present technology is directed to devices, systems, and methods for cardiac valve repair, and more particularly to valve repair devices with coaptation structures.

### BACKGROUND

Proper functioning of the mitral valve can be affected by mitral valve regurgitation, mitral valve prolapse, and/or mitral valve stenosis. Mitral valve regurgitation can occur when the leaflets of the mitral valve fail to coapt into apposition at peak contraction pressures such that blood leaks from the left ventricle into the left atrium. Several structural factors may affect the proper closure of the mitral valve leaflets. For example, an enlarged mitral annulus caused by dilation of heart muscle may prevent proper coaptation of the leaflets during systole. Other conditions involve a stretch or tear in the chordae tendineae—the tendons connecting the papillary muscles to the inferior side of the mitral valve leaflets—which may also affect proper closure of the mitral annulus. A ruptured chordae tendineae, for example, may cause a valve leaflet to prolapse into the left atrium due to inadequate tension on the leaflet. Abnormal backflow can also occur when the papillary muscles are compromised (e.g., due to ischemia) such that the affected papillary muscles do not contract sufficiently to effect proper closure during systole.

Mitral valve prolapse can occur when the mitral leaflets abnormally bulge up into the left atrium, which can also lead to mitral valve regurgitation. Normal functioning of the mitral valve may also be affected by mitral valve stenosis, or a narrowing of the mitral valve orifice, which impedes of filling of the left ventricle during diastole.

Mitral valve regurgitation is often treated using diuretics and/or vasodilators to reduce the amount of blood flowing back into the left atrium. Other treatment methods, such as surgical approaches (open and intravascular), have also been used to either repair or replace the native mitral valve. For example, cinching or resecting portions of the dilated annulus are typical repair approaches. Cinching of the annulus has been accomplished by implanting annular or peri-annular rings which are generally secured to the annulus or surrounding tissue. Other repair procedures have also involved suturing or clipping of the valve leaflets into partial apposition with one another. Alternatively, more invasive procedures replace the entire valve with mechanical valves or biological tissue. These invasive procedures are conventionally done through large open thoracotomies and are thus very painful, have significant morbidity, and require long recovery periods.

However, with many repair and replacement procedures, the durability of the devices or improper sizing of annuloplasty rings or replacement valves may cause complications. Moreover, many of the repair procedures depend upon the skill of the cardiac surgeon since poorly or inaccurately placed sutures may affect the success of procedures.

Compared to other cardiac valves, the mitral valve presents unique challenges because portions of the mitral valve annulus have limited radial support from surrounding tissue and the mitral valve has an irregular, unpredictable shape. For example, the anterior wall of the mitral valve is bound by only a thin wall separating the mitral valve annulus from the inferior portion of the aortic outflow tract. As a result, significant radial forces on the mitral valve annulus are not acceptable as they could lead to collapse of the inferior portion of the aortic tract with potentially fatal consequences. Another challenge of the mitral valve anatomy is that the maze of chordae tendineae in the left ventricle makes navigating and positioning a deployment catheter much more difficult compared to other heart valves. Given the difficulties associated with current procedures, there remains the need for simple, effective, and less invasive devices and methods for treating dysfunctional heart valves. Additionally, since it is also difficult to deliver devices to the mitral valve, there also remains the need for effective and less invasive delivery systems to deliver the implantable cardiac devices to the mitral valve.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure.

FIGS. 1A and 1B are a top view and a side view, respectively, of an implantable device that can be implanted within a heart of a subject in accordance with embodiments of the present technology.

FIGS. 2A-2C are different side views, and FIG. 2D is a top view, of an implantable device that can be implanted within a heart of a subject in accordance with additional embodiments of the present technology.

FIGS. 3A and 3B are a side view and a top view, respectively, of an atrial-fixation member of the implantable device of FIGS. 2A-2D in accordance with embodiments of the present technology.

FIGS. 4A and 4B are an enlarged side view and an enlarged perspective view of an attachment portion of the atrial-fixation member of FIGS. 2A-2D in accordance with embodiments of the present technology.

FIG. 5 is a side view of the atrial-fixation member of FIGS. 2A-2D including a posterior fabric member in accordance with embodiments of the present technology.

FIG. 6 is a side view of a coaptation member of the implantable device of FIGS. 2A-2D configured in accordance with embodiments of the present technology.

FIG. 7 is an enlarged side view of the coaptation member of FIGS. 2A-2D configured in accordance with embodiments of the present technology.

FIG. 8A is a side view of a clip of the implantable device of FIGS. 2A-2D configured in accordance with embodiments of the present technology. FIG. 8B is a side view of the implantable device of FIGS. 2A-2D without a coaptation covering over the coaptation member in accordance with embodiments of the present technology.

FIG. 9 is an enlarged side view of the implantable device of FIGS. 2A-2D in accordance with embodiments of the present technology.

FIGS. 10A and 10B are a side view and a top view of an implantable device that can be implanted within a heart of a subject in accordance with additional embodiments of the present technology.

FIGS. 11A and 11B are a side view and a top view of an atrial-fixation member of the implantable device of FIGS. 10A and 10B configured in accordance with embodiments of the present technology.

FIG. 12 is a side cross-sectional view of the implantable device of FIGS. 10A-10B implanted at a mitral valve in accordance with embodiments of the present technology.

FIG. 13 is a top view of an atrial-fixation member configured in accordance with additional embodiments of the present technology.

FIGS. 14A and 14B are a side view and a top view of an implantable device that can be implanted within a heart of a subject in accordance with additional embodiments of the present technology.

FIGS. 15A and 15B are a side view and a perspective top view of an atrial-fixation member of the implantable device of FIGS. 14A and 14B configured in accordance with embodiments of the present technology.

FIGS. 16A-16C are side views of a coaptation member of the implantable device of FIGS. 14A and 14B with a covering removed in accordance with embodiments of the present technology.

FIG. 17 is a side cross-sectional view of the implantable device of FIGS. 14A-14B implanted at a mitral valve in accordance with embodiments of the present technology.

FIG. 18 is a side view of a coaptation member and an associated clip assembly configured in accordance with embodiments of the present technology.

FIG. 19A is a side view of a clip assembly configured in accordance with additional embodiments of the present technology. FIG. 19B is a perspective side view of the clip assembly of FIG. 19A showing only a back member and an attachment member of the clip assembly in accordance with embodiments of the present technology.

FIG. 20 is an enlarged perspective side view of the coaptation member of the implantable device of FIGS. 14A and 14B including the clip assembly of FIGS. 19A and 19B in accordance with embodiments of the present technology.

FIGS. 21A-21C are side views illustrating the sequential attachment of a first fabric layer to a clip member of the clip assembly of FIGS. 19A and 19B in accordance with embodiments of the present technology.

FIGS. 22A and 22B are a perspective side view and a side view, respectively, illustrating the sequential attachment of a second fabric layer to the clip member of the clip assembly of FIGS. 19A and 19B in accordance with embodiments of the present technology.

### DETAILED DESCRIPTION

The present technology is directed to cardiac valve repair devices and associated systems and methods. In some embodiments, for example, cardiac valve repair devices (also referred to herein as “mitral valve repair devices,” “coaptation assist devices,” “implant devices,” and iterations thereof) include a coaptation member (also referred to as a “coaptation structure,” “baffle,” “intravalvular body,” “intermediate structure,” and iterations thereof) configured to be positioned between native valve leaflets of a cardiac valve. The coaptation member can be coupled to an atrial-fixation member (also referred to as a “brim,” “anchoring structure,” “fixation member,” and iterations thereof) that anchors to cardiac tissue within the atrium and/or provides a platform for ingrowth to hold the coaptation member in place.

In some embodiments, the coaptation member can comprise an inner portion having a coaptation surface configured to coapt with a first native leaflet (e.g., an anterior leaflet of a mitral valve) during systole and an outer portion configured to displace at least a portion of a second native leaflet (e.g., a posterior leaflet of the mitral valve). The atrial-fixation member can comprise a plurality of interconnected struts having a circumferential U-like shape about a flow axis of the cardiac valve. The interconnected struts can define a brim portion and a pair of connection portions. The atrial-fixation member can extend upward from the coaptation member relative to the flow axis. The connection portions can be coupled to the coaptation member, and the brim portion can be configured to press against cardiac tissue above the first native leaflet proximate to a native valve annulus of the cardiac valve. In some aspects of the present technology, the atrial-fixation member is configured not to press against cardiac tissue above the second native leaflet proximate to the native valve annulus.

In some embodiments, the atrial-fixation member can comprise a plurality of interconnected struts having a curved shape about a flow axis of the cardiac valve, and can be connected to the coaptation member via a pair of arm members extending therebetween. The atrial-fixation member can be configured to press against cardiac tissue above the first native leaflet proximate to the native valve annulus of the cardiac valve. The arm members can each have (i) a first portion that extends upward from the coaptation member relative to the flow axis and (ii) a second portion that curves downward toward the atrial-fixation member relative to the flow axis.

In some embodiments, a clip assembly can be coupled to the coaptation member and configured to secure the second native leaflet. The clip assembly can include: (i) a back member, (ii) a clip member having an arm portion and a root portion, (iii) a threaded member coupled to the back member, and (iv) an actuation member coupled to the threaded member. The root portion of the clip portion can be pivotably coupled to the back member, and the threaded member can be configured to rotate relative to the back member. The root portion can define a slot, and the actuation member can include a projection extending at least partially into the slot. The threaded member can be actuated to move the clip between an open position used to capture the second native leaflet and a closed position that secures the second native leaflet between the clip member and the coaptation member. More specifically, the threaded member can be rotated (e.g., via a component of an associated delivery system) in a first direction to drive the actuation member in a first direction (e.g., upward) along the threaded member to drive the arm portion to pivot away from the outer portion of the coaptation member toward the open position. Conversely, the threaded member can be rotated in a second direction to drive the actuation member in a second direction (e.g., downward) along the threaded member to drive the arm portion to pivot toward the outer portion of the coaptation member toward the closed position.

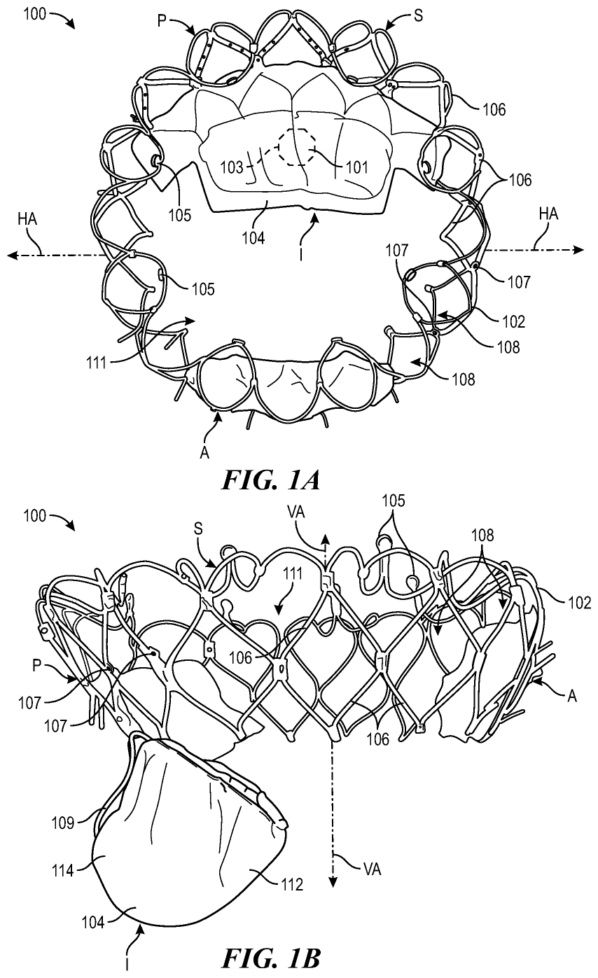

Specific details of several embodiments of the technology are described below with reference to FIGS. 1A-22B. Although many of the embodiments are described below with respect to implant devices, systems, and methods for repair of a native mitral valve, other applications and other embodiments in addition to those described herein are within the scope of the technology. For example, the present technology may be used at other target sites, like the tricuspid valve, the pulmonary valve, and/or the aortic valve. Additionally, several other embodiments of the technology can have different configurations, components, or procedures than those described herein, and features of the embodiments shown can be combined with one another. A person of ordinary skill in the art, therefore, will accordingly understand that the technology can have other embodiments with additional elements, or the technology can have other embodiments without several of the features shown and described below with reference to FIGS. 1A-22B. In some instances, well-known structures and techniques often associated with cardiac implants and prosthetic heart valves have not been shown in detail so as not to obscure the present technology. The terminology used in the description presented below is intended to be interpreted in its broadest reasonable manner, even though it is being used in conjunction with a detailed description of certain specific embodiments of the disclosure.

The accompanying Figures depict embodiments of the present technology and are not intended to be limiting of its scope. The sizes of various depicted elements are not necessarily drawn to scale, and these various elements can be arbitrarily enlarged to improve legibility. Component details can be abstracted in the Figures to exclude details such as position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the spirit or scope of the present technology.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” etc., are not meant to limit the referenced component to use in a specific orientation. It will be appreciated that such designations refer to the orientation of the referenced component as illustrated in the Figures; the systems of the present technology can be used in any orientation suitable to the user.

With regard to the terms “distal” and “proximal” within this description, unless otherwise specified, the terms can reference a relative position of the portions of a valve repair device and/or an associated delivery device with respect to an operator and/or a location in the vasculature or heart. For example, in referring to a delivery catheter suitable to deliver and position various valve repair devices described herein, “proximal” can refer to a position closer to the operator of the device or an incision into the vasculature, and “distal” can refer to a position that is more distant from the operator of the device or further from the incision along the vasculature (e.g., the end of the catheter). With respect to a heart valve repair device, the terms “proximal” and “distal” can refer to portions of the device relative to the native annulus. For example, “proximal” can refer to an upstream portion of the device spaced apart from the native annulus, and “distal” can refer to a downstream position at or proximate to the native annulus.

Further, as used herein, the designations “forward,” “rearward,” “upward,” “downward,” “top,” “bottom,” etc., are not meant to limit the referenced component to use in a specific orientation. It will be appreciated that such designations refer to the orientation of the referenced component as illustrated in the Figures. However, the systems of the present technology can be used in any orientation suitable to the user.

FIGS. 1A and 1B are a top view and a side view, respectively, of an implantable device 100 that can be implanted within a heart of a subject (e.g., a human patient) in accordance with embodiments of the present technology. Referring to FIGS. 1A and 1B together, in the illustrated embodiment the implantable device 100 is a valve repair device having an atrial-fixation member 102 (also referred to as an “anchoring member” or a “brim”) and a coaptation member 104 (also referred to as a “baffle”) extending from the atrial-fixation member 102 in a downstream direction. The atrial-fixation member 102 is configured to anchor the implantable device 100 to cardiac tissue proximate to a native mitral valve annulus and position the coaptation member 104 at a desired location with respect to the native valve anatomy of the heart. The coaptation member 104 is configured to displace at least a portion of one or more native leaflets of a cardiac valve and create a prosthetic coaptation surface for at least a portion of one or more of the other native leaflets of the cardiac valve. For example, when the implantable device 100 is deployed across the mitral valve annulus, the coaptation member 104 may extend in front of a central portion of the posterior leaflet (i.e., P2 of the posterior leaflet), pushing the posterior leaflet back toward the ventricular wall, such that the coaptation member 104 is positioned to coapt with the anterior leaflet during systole. The implantable device 100 is configured relative to a flow axis VA (FIG. 1B) in the direction of blood flow from the atrium to the ventricle and a transverse axis HA (FIG. 1A) at an angle (e.g., orthogonal) to the flow axis VA. The implantable device 100 has a posterior side portion P (e.g., a first side portion), an anterior side portion A (e.g., a second side portion), a superior end portion S (e.g., a first end portion), and an inferior end portion I (e.g., a second end portion).

In some embodiments, the implantable device 100 can include some features generally similar or identical to the implantable devices described in (i) U.S. patent application Ser. No. 16/044,447, titled “PROSTHETIC LEAFLET DEVICE,” and filed Jul. 24, 2018, (ii) International Patent Application No. PCT/US2018/061126, titled “LEAFLET EXTENSION FOR CARDIAC VALVE LEAFLET,” and filed Nov. 14, 2018, (iii) U.S. patent application Ser. No. 16/745,246, titled “IMPLANTABLE COAPTATION ASSIST DEVICES WITH SENSORS AND ASSOCIATED SYSTEMS AND METHODS,” and filed Jan. 16, 2020, (iv) U.S. patent application Ser. No. 16/817,464, titled “CARDIAC VALVE REPAIR DEVICES WITH ANNULOPLASTY FEATURES AND ASSOCIATED SYSTEMS AND METHODS,” and filed Mar. 12, 2020, (v) U.S. patent application Ser. No. 17/027,681, titled “VALVE REPAIR DEVICES WITH COAPTATION STRUCTURES AND MULTIPLE LEAFLET CAPTURE CLIPS,” and filed Sep. 21, 2020, and/or (vi) U.S. patent application Ser. No. 17/184,113, titled “DELIVERY SYSTEMS FOR CARDIAC VALVE DEVICES, AND ASSOCIATED METHODS OF OPERATION,” and filed Mar. 5, 2021, each of which are incorporated herein by reference in their entirety. Any of several prosthetic valve repair or replacement devices could similarly be used with delivery systems in accordance with the present technology, including complete mitral valve replacement devices. And, in addition to mitral valve devices, other valve repair or replacement devices could be delivered to the tricuspid, aortic, and pulmonic valves using delivery systems in accordance with the present invention.

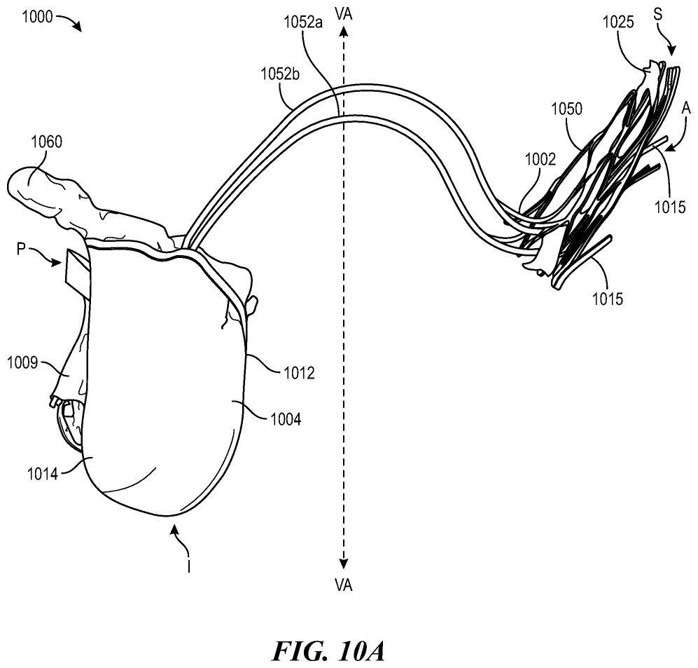

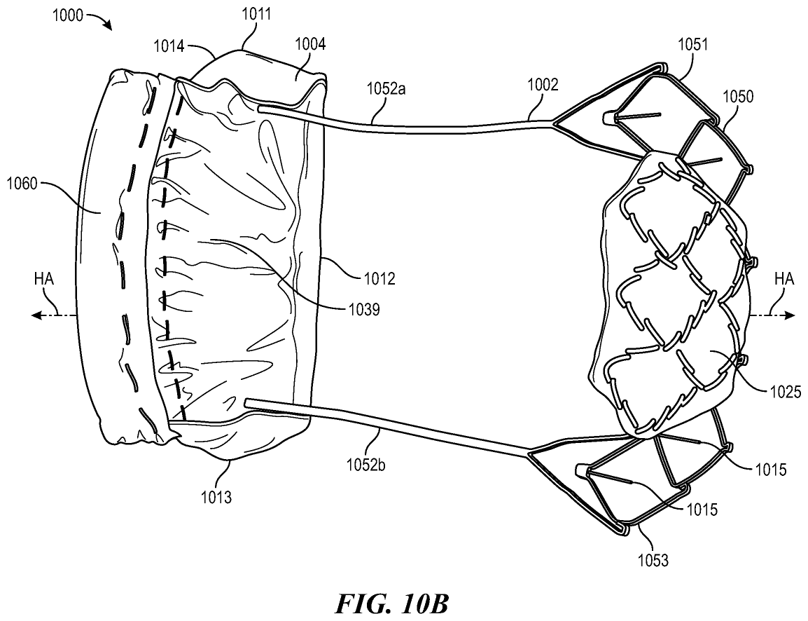

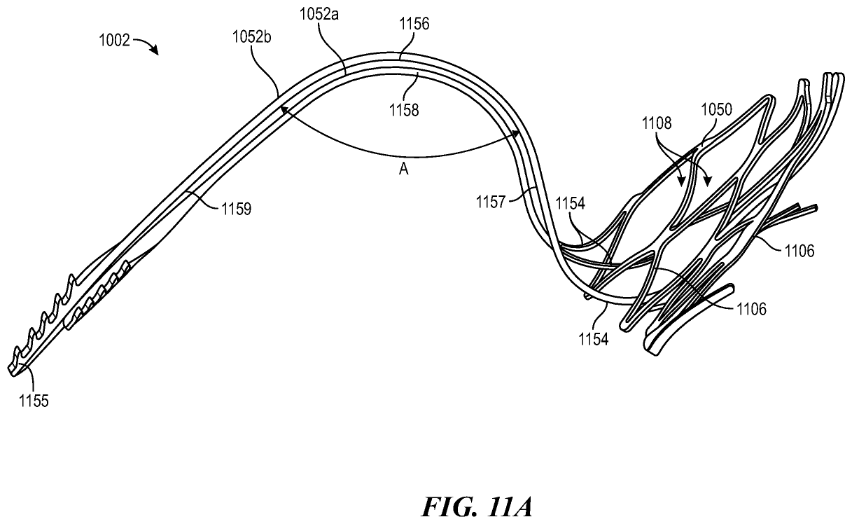

The atrial-fixation member 102 can be formed of a mesh, such as a braid or laser-cut stent-like structure, including a plurality of interconnected wires or struts 106 which together define a plurality of openings or cells 108 (e.g., diamond-shaped openings) arranged in one or more rows. The struts 106 can be configured to self-expand from a collapsed delivery state (not shown) to an expanded deployed state shown in FIGS. 1A and 1B. The struts 106 can be formed of any biocompatible material such as, for example, stainless steel, nickel-titanium alloys (e.g., nitinol), and/or other suitable stent materials. The atrial-fixation member 102 can have a generally circular, oval, or D-like shape in the deployed state and define an open central lumen 111 (also referred to as an “opening”) that allows blood to pass therethrough along the flow axis VA. In the illustrated embodiment, the atrial-fixation member 102 extends entirely circumferentially about the flow axis VA. When the implantable device 100 is configured to repair a native mitral valve, the atrial-fixation member 102 can be shaped to conform to the walls of the left atrium just above the mitral annulus to secure the implantable device 100 to the supra-annular tissue. After a period of time post-implantation (e.g., 3 days, 2 weeks, 1 month, 2 months), the atrial-fixation member 102 or portions thereof become covered by a layer of tissue, and this tissue ingrowth adheres the implantable device 100 permanently to the atrial wall. In some embodiments, as described in greater detail below with reference to FIGS. 10A-17, the atrial-fixation member 102 has a U-like or other shape that does not extend fully around the circumference of the native valve. In some embodiments, the atrial-fixation member 102 may also or alternatively include one or more portions that press against sub-annular tissue to provide sub-annular device fixation.

In some embodiments, the atrial-fixation member 102 can include connectors 105 that are configured (e.g., sized, shaped, and/or positioned) to engage with a mating feature on the delivery system, such as any of the mating features described and illustrated in U.S. patent application Ser. No. 17/184,113, titled “DELIVERY SYSTEMS FOR CARDIAC VALVE DEVICES, AND ASSOCIATED METHODS OF OPERATION,” and filed Mar. 5, 2021, which is incorporated herein by reference in its entirety. As shown in FIG. 1B, for example, the connectors 105 can extend from the struts 106 such that the connectors 105 are positioned near or at the superior end portion S of the implantable device 100. In some embodiments, the atrial-fixation member 102 includes one or more eyelets 107 configured to receive one or more tendons that aids in packing (e.g., compressing), delivering, orienting, and/or retrieving the implantable device 100. For example, the tendons can help facilitate cinching (e.g., radially compressing) of the atrial-fixation member 102. The eyelets 107 can be metal portions of the atrial-fixation member 102, or can be separate filaments/wires forming loops and attached to the atrial-fixation member 102.

As shown in FIGS. 1A and 1B, the coaptation member 104 extends away from a downstream end portion of the atrial-fixation member 102 along the flow axis VA and at least a portion of the coaptation member 104 extends radially inward from the atrial-fixation member 102 into the central lumen 111 to approximate a closed position of a native leaflet. The coaptation member 104 can be substantially stationary (e.g., little to no movement) during cardiac cycles such that the position of the coaptation member 104 relative to the atrial-fixation member 102 is at least substantially fixed in the deployed state. Thus, unlike native leaflets that move back and forth to open and close the native valve, the coaptation member 104 remains stationary during diastole and systole.

The coaptation member 104 can have an anterior portion 112 (FIG. 1B) with a smooth, atraumatic surface for coapting with at least a portion of one or more native leaflets and a posterior portion 114 (FIG. 1B) configured to displace and, optionally, engage at least a portion of another native leaflet. The coaptation member 104 can be made from a plurality of struts that form a basket-like or frame-like structure (e.g., a mesh structure, a laser cut stent frame) with an at least partially hollow interior and a covering (e.g., a fabric) extending over at least a portion of the struts to provide a smooth suitable surface for coaptation at the anterior portion 112. The covering may also extend over the struts along the posterior portion 114 and between the anterior and posterior portions 112, 114 in a manner that forms lateral sidewalls. The atrial-fixation member 104 or portions thereof can be integral with the atrial-fixation member 102 such that, for example, the coaptation member 104 is manufactured from the same frame including the struts 106. In other embodiments, the atrial-fixation member 104 can be a separate structure that is connected to a portion of the atrial-fixation member 102 during manufacturing. In some embodiments, the atrial-fixation member 104 can include a biocompatible foam which is attached to the structure of the atrial-fixation member 104 and/or to the atrial-fixation member 102.

In the illustrated embodiment, the atrial-fixation member 104 further includes a normally-closed clip 109 (obscured in FIG. 1A) depending from its posterior surface which can be opened to extend behind the native leaflet the coaptation member 104 displaces. The clip 109 may grasp the native leaflet and/or engage sub-annular cardiac tissue for sub-annular stabilization of the implantable device 100. In some embodiments, for example, the clip 109 reaches under the central portion (i.e., P2) of the posterior leaflet up to the sub-annular space. A tendon (made of suture or nitinol wire) can actuate the clip 109 by way of a lever attached to the clip 109. The lever may be a nitinol wire or laser cut nitinol or Co-Cr sheet.

As shown in FIG. 1A, the atrial-fixation member 104 can further include a delivery attachment member 103 (shown in broken lines) positioned within the hollow interior of the atrial-fixation member 104. The delivery attachment member 103 can be a threaded nut or other type of connector configured to mate with a corresponding portion (e.g., a screw) of the delivery system, such as such as portions of the delivery systems described and illustrated in U.S. patent application Ser. No. 17/184,113, titled “DELIVERY SYSTEMS FOR CARDIAC VALVE DEVICES, AND ASSOCIATED METHODS OF OPERATION,” and filed Mar. 5, 2021, which is incorporated herein by reference in its entirety. In some embodiments, the delivery attachment member 103 is accessible via a flap or opening 101 (FIG. 1A) formed in the atrial-fixation member 104 (e.g., in portion of the baffle facing the superior end portion S of the implantable device 100).

The implantable device 100 may be inserted via a femoral vein sheath to traverse the inferior vena cava to the right atrium. The implantable device 100 is then inserted into the left atrium via a puncture of the interatrial septum. In several applications, the implantable device 100 is delivered to a target location within the mitral valve to function properly. This means appropriate positioning along the flow axis VA, correct radial positioning relative to the central axis of the valve, correct rotational orientation to specific landmarks such as the middle (P2) portion of the native posterior leaflet, and correct angular positioning relative to the flow axis and the transverse axis. In some embodiments, the implantable device 100 may also be repositioned during the delivery process to, for example, correct for misalignment or inappropriate positioning. During deployment and release of the implantable device 100, the delivery system can retain the implantable device 100 in a stationary position at the desired location and in the desired orientation relative to the native valve. Furthermore, the delivery system may be configured to allow the implantable device 100 to be re-sheathed, repositioned, and/or removed before being released from the delivery system. Delivery systems of the present technology can achieve all the above-mentioned advantages in a user-friendly system. Additionally, several embodiments of delivery systems in accordance with the present technology have a small overall diameter, such as approximately 15 to 30 French.

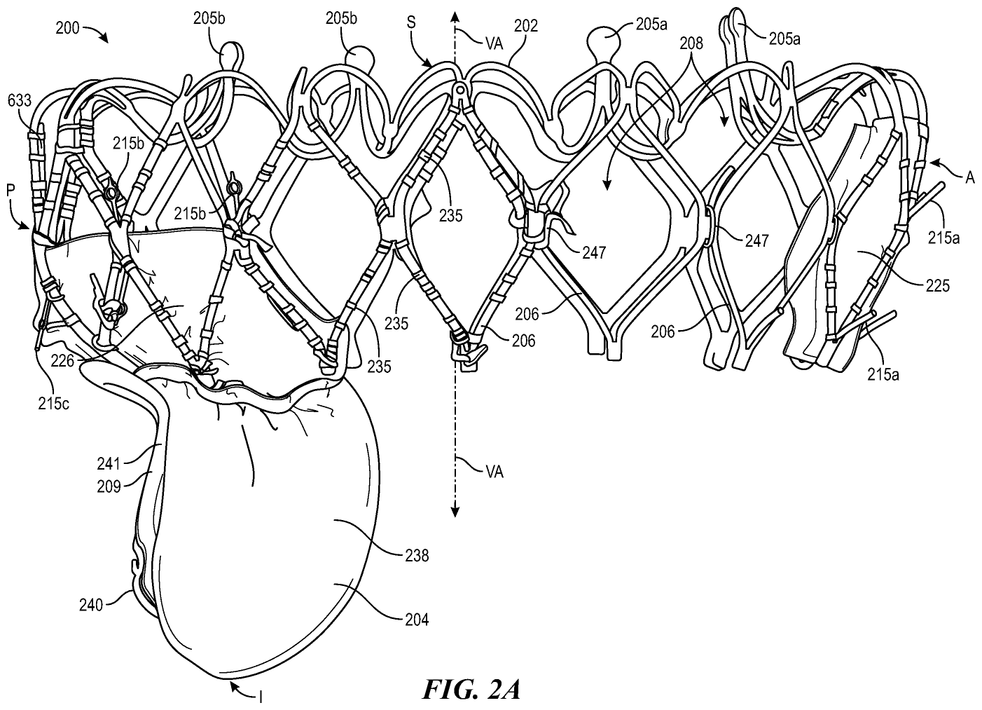

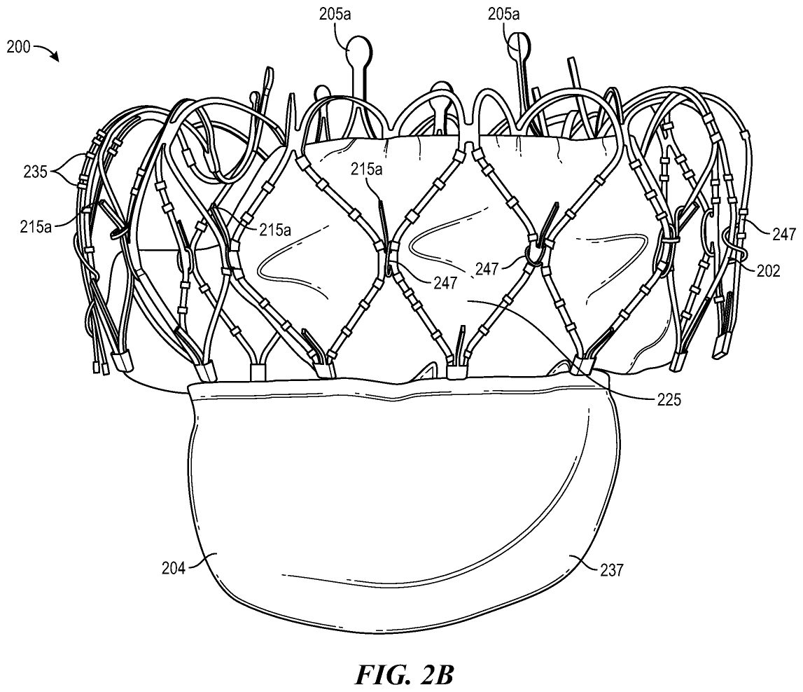

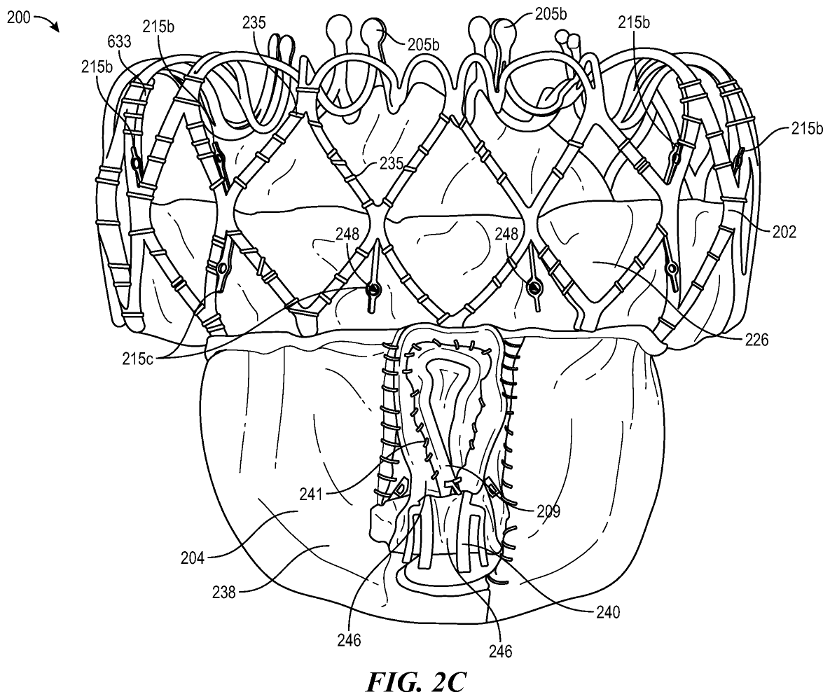

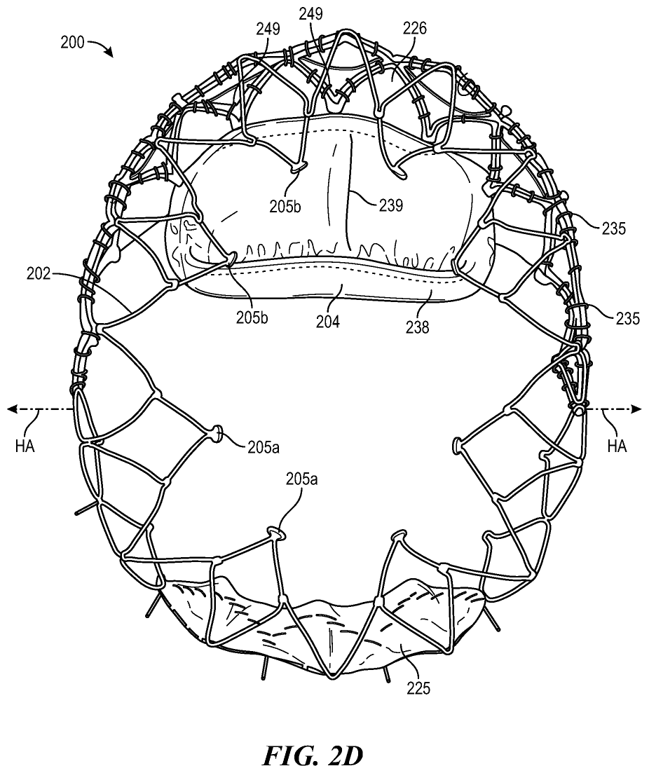

FIGS. 2A-2C are different side views (e.g., an anterior-posterior side view, an anterior or commissure-commissure side view, and a posterior side view, respectively) and FIG. 2D is a top view (e.g., an en face view) of an implantable device 200 that can be implanted within a heart of a subject (e.g., a human patient) in accordance with embodiments of the present technology. The implantable device 200 can include some features that are at least generally similar in structure and function, or identical in structure and function, to the corresponding features of the implantable device 100 described in detail above with reference to FIGS. 1A and 1B, and can operate in a generally similar or identical manner to the implantable device shown in FIGS. 1A and 1B.

For example, referring to FIGS. 2A-2D, in the illustrated embodiment the implantable device 200 is a valve repair device having an atrial-fixation member 202 and a coaptation member 204 extending from the atrial-fixation member 202 in a downstream direction. The atrial-fixation member 202 is configured to anchor the implantable device 200 to cardiac tissue proximate to a native mitral valve annulus and position the coaptation member 204 at a desired location with respect to the native valve anatomy of the heart. The implantable device 200 is designed to eliminate regurgitation by restoring physiologic coaptation in a diseased mitral valve. The coaptation member 204 can be covered in a covering of biocompatible material (e.g., expanded polytetrafluoroethylene (ePTFE)) and shaped (e.g., contoured) to fill a regurgitant orifice of the mitral valve from the posterior side and provide a new coaptation surface for the native leaflets of the mitral valve. The atrial-fixation member 202 can be flexible and can provide additional fixation and supra-annular stabilization. In the illustrated embodiment, the implantable device 200 further includes a posterior clip 209 (obscured in FIGS. 2B and 2D) that can orient the implantable device 200 and provide sub-annular fixation.

The implantable device 200 is configured relative to a flow axis VA (FIG. 2A) in the direction of blood flow from the atrium to the ventricle and a transverse axis HA (FIG. 2D) at an angle (e.g., orthogonal) to the flow axis VA. As shown in FIG. 2A, the implantable device 100 has a posterior side portion P (e.g., a first side portion), an anterior side portion A (e.g., a second side portion), a superior end portion S (e.g., a first end portion), and an inferior end portion I (e.g., a second end portion).

Referring to FIG. 2A, the atrial-fixation member 202 can be formed of a mesh, such as a braid or laser-cut stent-like structure, including a plurality of interconnected wires or struts 206 which together define a plurality of openings or cells 208 (e.g., diamond-shaped openings) arranged in one or more rows. The struts 206 can be configured to self-expand from a collapsed delivery state (not shown) to an expanded deployed state shown in FIGS. 2A and 2B.

Referring to FIGS. 2A-2C, the atrial-fixation member 202 can include fixation features in the form of cleats. For example, the atrial-fixation member 202 can include upward facing anterior cleats 215a (e.g., nine upward facing anterior cleats) and upward facing posterior cleats 215b (e.g., four upward facing posterior cleats) to inhibit or even prevent migration of the implantable device 200 into the left atrium. Similarly, the atrial-fixation member 202 can include downward facing posterior cleats 215c (e.g., four downward facing posterior cleats) to inhibit or even prevent slipping of the implantable device 200 into the left atrium. In some embodiments, the downward facing posterior cleats 215c can originate from (e.g., extend from) the coaptation member 204.

Referring to FIGS. 2A-2D, the atrial-fixation member 202 can include anterior connectors 205a (e.g., tab features) and posterior connectors 205b that are configured (e.g., sized, shaped, and/or positioned) to engage with a mating feature on an associated delivery system, such as any of the mating features described and illustrated in U.S. patent application Ser. No. 17/184,113, titled “DELIVERY SYSTEMS FOR CARDIAC VALVE DEVICES, AND ASSOCIATED METHODS OF OPERATION,” and filed Mar. 5, 2021, which is incorporated herein by reference in its entirety. The connectors 205 can be extend from the struts 206 such that the connectors 205 are positioned at or near the superior end portion S (FIG. 2A) of the implantable device 200. In some embodiments, as best seen in FIG. 2A, the posterior connectors 205b are shorter than the anterior connectors 205a to facilitate their release from the delivery system first. The longer anterior connectors 205a facilitate their release after the posterior connectors 205b. The atrial-fixation member 202 can include four each of the posterior connectors 205b and the anterior connectors 205a, or a different number.

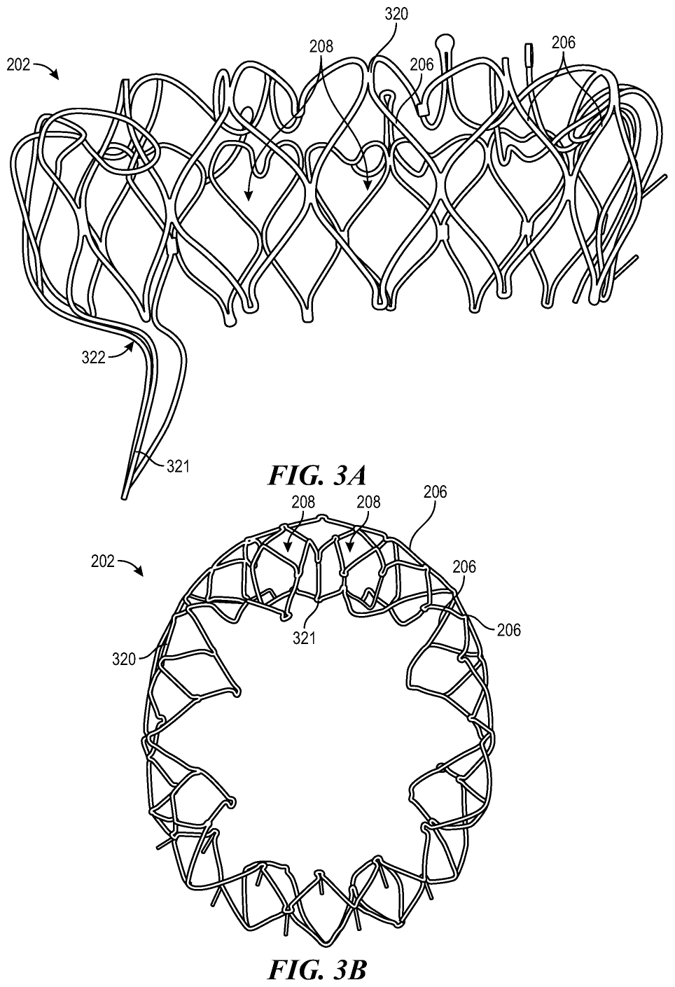

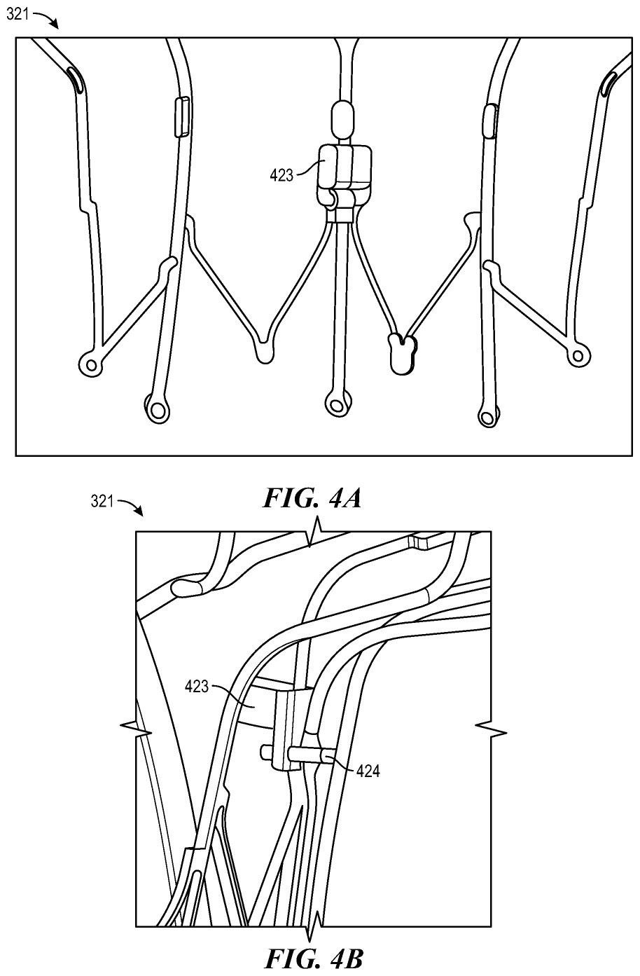

FIGS. 3A and 3B are a side view and a top view, respectively, of the atrial-fixation member 202 of the implantable device 200 in accordance with embodiments of the present technology. Referring to FIGS. 3A and 3B, the atrial-fixation member 202 includes an atrial-fixation portion 320 and an attachment portion 321 (e.g., a “brim-baffle extension”) extending downstream from the atrial-fixation portion 320 for connection to the coaptation member 204 (FIGS. 2A-2D). The atrial-fixation member 202 can be made by shaping a tubular nitinol stent. The atrial-fixation portion 320 can have a 16-column diamond stent pattern (e.g., including 16 columns of the openings 208 arranged circumferentially about the flow axis VA) that extends entirely circumferentially about the flow axis VA. In other embodiments, the atrial-fixation member 202 can have other configurations and/or another number of columns. In some embodiments, the struts 206 have a width from about 0.013-0.017 inch and the struts 206 are electropolished to have a final thickness of about 0.017 inch. Referring to FIG. 3A, the attachment portion 321 can have a jog 322 to facilitate better posterior fixation and minimize the area of the coaptation member 204 (FIGS. 2A-2D) while still achieving required protrusion of the coaptation member 204 for coaptation with an opposing native leaflet and/or to avoid interference with portions of the subannular native anatomy.

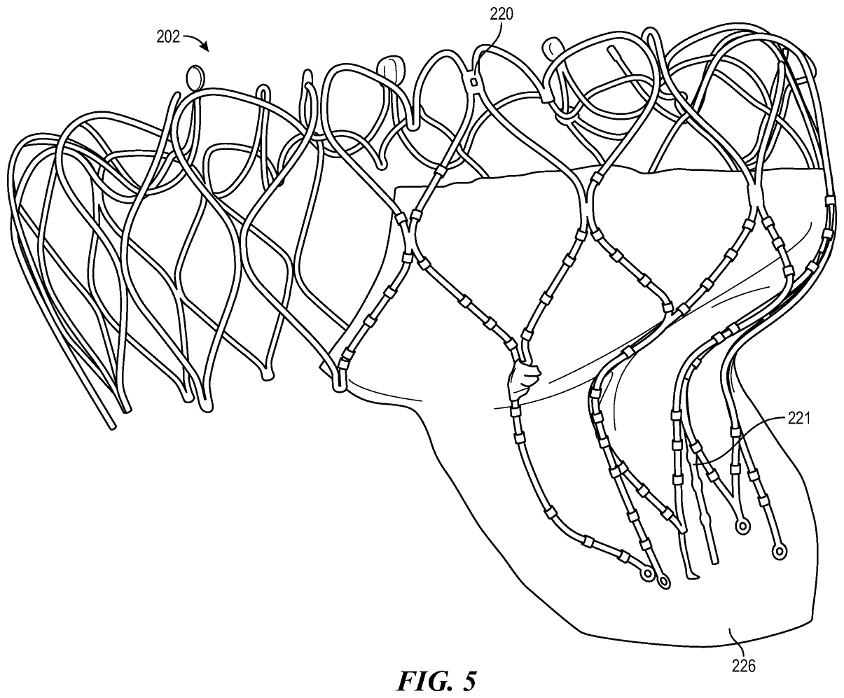

In some embodiments, the attachment portion 321 can include one or more features configured to be secured to an associated delivery system. FIGS. 4A and 4B, for example, are an enlarged side view and an enlarged perspective view of the attachment portion 321 of the atrial-fixation member 202 in accordance with embodiments of the present technology. Referring to FIGS. 4A and 4B, the attachment portion 421 can include a nut 423 (e.g., a M1 nut) secured thereto (e.g., via a rivet 424 shown in FIG. 4B) to provide a feature for attachment of the attachment portion 321 and coaptation member 204 (FIGS. 2A-2D) to the delivery system.

Referring to FIGS. 2A-2D, the implantable device 200 can include one or more portions of fabric attached to the atrial-fixation member 202 to facilitate long term tissue ingrowth into the structure of the atrial-fixation member 202. For example, in the illustrated embodiment the implantable device 200 includes an anterior fabric member 225 attached along a portion of the anterior side portion A (FIG. 2A) of the atrial-fixation member 202 and a posterior fabric member 226 attached along a portion of the posterior side portion P (FIG. 2A) of the of the atrial-fixation member 202 (e.g., proximate and/or adjacent to the coaptation member 204). The fabric members 225, 226 can comprise polyethylene terephthalate (PET) and/or another suitable material that facilitates long term tissue ingrowth, and can be secured to the atrial-fixation member 202 via sutures and/or other suitable fasteners. In some embodiments, the posterior fabric member 226 extends along the attachment portion 321 (FIGS. 3A and 3B) of the atrial-fixation member 202 behind the clip 209 to facilitate long term tissue ingrowth into the structure under the clip 209. For example, FIG. 5 is a side view of the atrial-fixation member 202 including the posterior fabric member 226 in accordance with embodiments of the present technology. In the illustrated embodiment, the posterior fabric member 226 extends substantially along/over the attachment portion 321 and at least partially along the atrial-fixation portion 320.

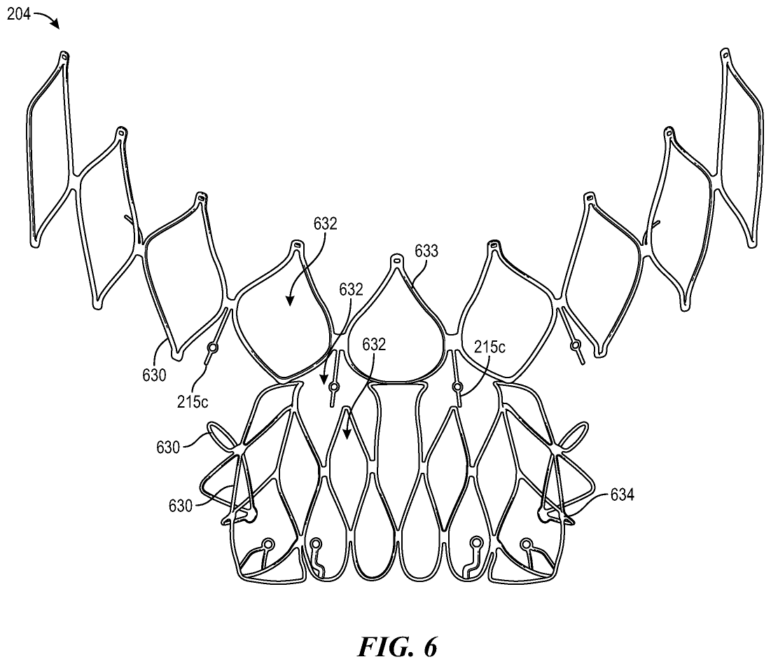

FIG. 6 is a side view of the coaptation member 204 of the implantable device 200 configured in accordance with embodiments of the present technology. The coaptation member 204 can be formed of a mesh, such as a braid or laser-cut stent-like structure, including a plurality of interconnected wires or struts 630 which together define a plurality of openings or cells 632. The struts 630 can be configured to self-expand from a collapsed delivery state (not shown) to an expanded deployed state shown in FIGS. 2A-2D. The coaptation member 204 can be made from a laser cut nitinol flat sheet and the struts 630 can have a thickness of between about 0.008-0.15 inch (e.g., about 0.012 inch).

In the illustrated embodiment, the coaptation member 204 includes an atrial portion 633 and a coaptation portion 634 extending downstream from the atrial portion 633. The atrial portion 633 can have a diamond stent pattern that generally matches the diamond stent pattern of the atrial-fixation portion 320 (FIGS. 3A and 3B) of the atrial-fixation member 202. The coaptation portion 634 is configured to define an at least partially hollow interior space when the implantable device 200 is in the deployed state. In the illustrated embodiment, the downward facing posterior cleats 215c extend from the atrial portion 633. Referring to FIGS. 2A-3B and FIG. 6, the atrial portion 633 of the coaptation member 204 can be secured to the atrial-fixation portion 320 of the atrial-fixation member 202 via, for example, sutures 235 shown in FIGS. 2A-2D. Specifically, the atrial portion 633 is best seen secured to the atrial-fixation member 202 in FIGS. 2A and 2C. The sutures 235 can be formed from ultra-high molecular weight polyethylene (UHMWPE) material and/or other suitably strong, biocompatible, and durable materials. In the illustrated embodiment, the diamond stent patterns of the atrial-fixation portion 320 and the atrial portion 633 are aligned such that the struts 206, 630, respectively, are adjacent one another and secured together via the sutures 235. The atrial portion 633 of the coaptation member can include nine of the diamond openings 632 such that the atrial portion 633 extends only partially about the atrial-fixation member 233 (e.g., proximate the posterior side portion P), or another number of the diamond openings 632. In some embodiments the coaptation portion 634 of the coaptation member 204 is secured to the attachment portion 221 of the atrial-fixation member 202 via suturing and/or another attachment technique.

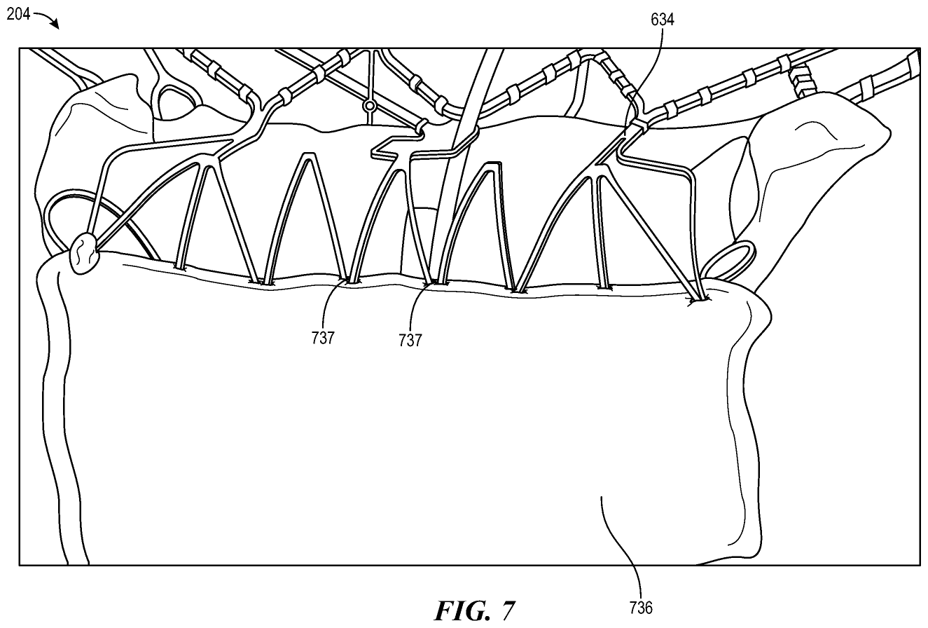

The coaptation portion 634 of the coaptation member 204 and the attachment portion 221 of the atrial-fixation member 202 can be covered with a coaptation covering, such a polyurethane (PU) foam and/or fabric layer to provide an atraumatic coaptation surface. For example, FIG. 7 is an enlarged side view of the coaptation member 204 configured in accordance with embodiments of the present technology. In the illustrated embodiment, a foam 736 is secured to the anterior portion of the coaptation portion 634 via sutures 737. The foam 736 can comprise a 1-millimeter-thick PU foam and the sutures 737 can be UHMWPE sutures and/or other sutures. Additionally referring to FIGS. 2A-2D, the foam 736 and the coaptation portion 634 can further be covered with a fabric layer 238, such as an ePTFE fabric having a thickness of about 0.6 millimeter. The foam 736 and the fabric layer 238 can together comprise a coaptation covering that provides an atraumatic coaptation surface. Referring to FIG. 2D, the fabric layer 238 can comprise a slit 239 at a superior (e.g., top) portion of the coaptation member 204 to allow for one or more components (e.g., a baffle screw, core plug, clip tendon) of an associated delivery system to attach to the coaptation member 204. For example, a portion of the clip 209 (FIGS. 2A and 2C) and the nut 423 (FIGS. 4A and 4B) can be accessed via the slit 239. The slit 239 can be configured to close after the coaptation member 204 is detached from the associated delivery system.

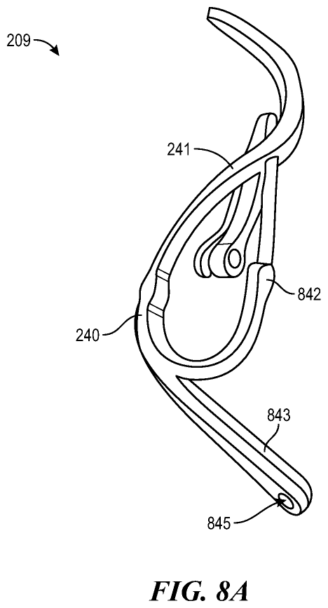

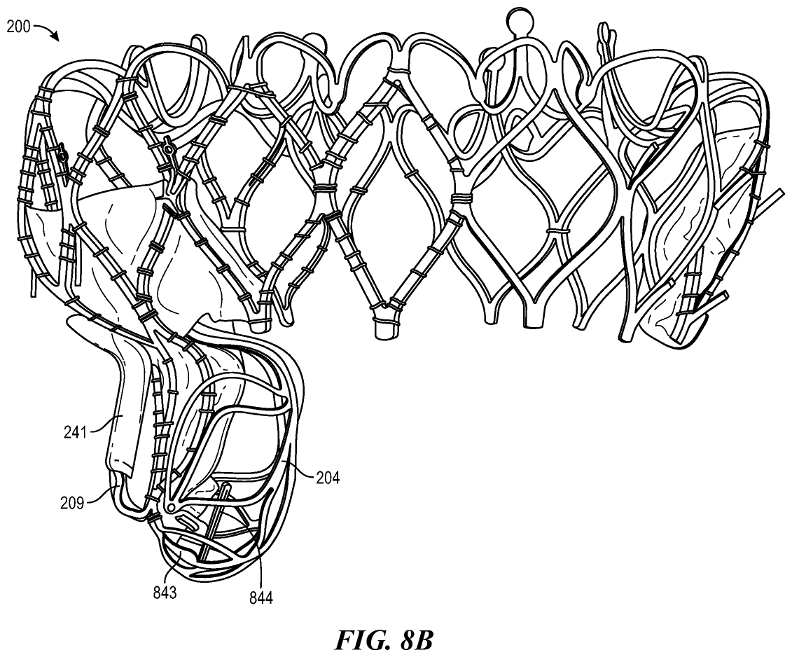

Referring to FIGS. 2A and 2C, the clip 209 includes a root 240 and an arm 241 extending from the root 240. FIG. 8A is a side view of the clip 209 configured in accordance with embodiments of the present technology. In the illustrated embodiment, the clip 209 further includes a back 842 extending from the root 240 and a lever 843 that facilitates actuation of the arm 241. The clip 209 can be made be made from a laser cut nitinol tube. FIG. 8B is a side view of the implantable device 200 without a coaptation covering (e.g., the foam 736 and fabric layer 238 shown in FIGS. 7 and 2A-2D, respectively) over the coaptation member 204 in accordance with embodiments of the present technology. Referring to FIGS. 8A and 8B, the back 842 of the clip 209 can be secured to the posterior side of the coaptation member 204 via, for example, suturing. A clip tendon suture 844 (e.g., a UHMWPE suture; FIG. 8B) can be attached to the lever 843 (e.g., at an attachment point 845 shown in FIG. 8A) for actuating the arm 241. The clip tendon suture 844 can be coupled to a component of an associated delivery system during delivery for actuating the clip 209. In some embodiments, during implantation of the implantable device 200, the clip 209 can be attached to the posterior back of the coaptation member 204 using the clip tendon suture 844. Referring to FIG. 2C, in some embodiments the clip 209 is at least partially covered with fabric 246 (e.g., PTFE and/or ePTFE) to promote tissue ingrowth into the structure for long term fixation. For example, the root 240 can be layered with the fabric 246 to provide for atraumatic interaction with the posterior leaflet free edge.

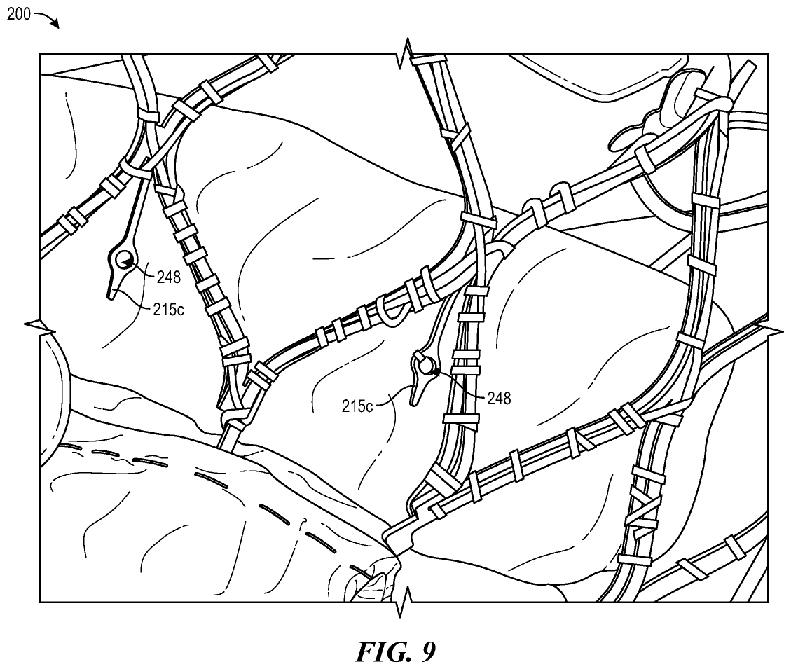

Referring again to FIGS. 2A-2D, the implantable device 200 can include features for receiving a removable cinching line for cinching the implantable device 200. In some aspects of the present technology, cinching allows for a reduction of the profile of the implantable device 200 to facilitate packing into a sleeve of an associated delivery system and/or for repositioning of the implantable device 200 after uncinching in the left atrium. For example, as best seen in FIGS. 2A and 2B, the implantable device 200 can include multiple loops 247 (e.g., UHMWPE suture loops) formed of suture and attached to an anterior portion of the atrial-fixation member 202. In the illustrated embodiment, the implantable device 200 includes twelve of the loops 247 and individual ones of the loops 247 are coupled to corresponding middle nodes of the diamond stent pattern of the atrial-fixation portion 320 (FIGS. 3A and 3B). As best seen in FIG. 2C, the implantable device 200 can further include multiple eyelets 248 formed in corresponding ones of the downward facing posterior cleats 215c. FIG. 9 is an enlarged side view of the implantable device 200 in accordance with embodiments of the present technology and further illustrating the eyelets 248 in the downward facing posterior cleats 215c. In the illustrated embodiment, the implantable device 200 can include four of the eyelets 248. In some embodiments, eyelets can additionally or alternatively be formed in the upward facing posterior cleats 215b for receiving a cinching line. As best seen in FIG. 2D, the implantable device 200 can further include multiple guide loops 249 (e.g., UHMWPE suture loops) connected to the posterior portion of the atrial-fixation member 202 inward of the posterior fabric member 226.

Referring to FIGS. 2A-2D, one or more cinching lines can traverse through the loops 247, the eyelets 248, and/or the guide loops 249 for cinching the implantable device 200. A cinching line extending through the loops 247 can cinch the anterior portion of the atrial-fixation member 202, the same or a different cinching line extending through the eyelets 248 can cinch the posterior portion of the atrial-fixation member 202 and radially pull in the downward facing posterior cleats 215c (and/or the upward facing posterior cleats 215b), and the same or a different cinching line extending through the guide loops 249 can allow for the posterior cleats 215b-c to be biased and/or pulled in radially. In some aspects of the present technology, this can inhibit or even prevent the cleats 215 from interacting with the sleeve of an associated delivery system during unsheathing.

FIGS. 10A and 10B are a side view (e.g., an anterior-posterior side view) and a top view (e.g., an en face view) of an implantable device 1000 that can be implanted within a heart of a subject (e.g., a human patient) in accordance with embodiments of the present technology. The implantable device 1000 can include some features that are at least generally similar in structure and function, or identical in structure and function, to the corresponding features of the implantable device 100 and/or the implantable device 200 described in detail above with reference to FIGS. 1A-9, and can operate in a generally similar or identical manner to the implantable devices shown in FIGS. 1A-9.

For example, referring to FIGS. 10A and 10B, in the illustrated embodiment the implantable device 1000 is a valve repair device having an atrial-fixation member 1002 (also referred to as an “anterior brim,” an “anterior-only brim,” an “anterior anchoring member” or a “suprannular anchoring member”) and a coaptation member 1004 extending from the atrial-fixation member 1002 in a downstream direction. The atrial-fixation member 1002 is configured to anchor the implantable device 1000 to cardiac tissue proximate to a native mitral valve annulus and position the coaptation member 1004 at a desired location with respect to the native valve anatomy of the heart. The implantable device 1000 is designed to eliminate regurgitation by restoring physiologic coaptation in a diseased mitral valve. The coaptation member 1004 can be covered in a biocompatible material (e.g., polytetrafluoroethylene (PTFE), expanded polytetrafluoroethylene (ePTFE)) and shaped (e.g., contoured) to fill a regurgitant orifice of the mitral valve from the posterior side and provide a new coaptation surface for the native leaflets of the mitral valve. The atrial-fixation member 1002 can be flexible and can provide additional fixation and supra-annular stabilization. In the illustrated embodiment, the implantable device 1000 further includes a posterior clip 1009 (obscured in FIG. 10B) that can orient the implantable device 1000 and provide sub-annular fixation.

The implantable device 1000 is configured relative to a flow axis VA (FIG. 10A) in the direction of blood flow from the atrium to the ventricle and a transverse axis HA (FIG. 10B) at an angle (e.g., orthogonal) to the flow axis VA. As shown in FIG. 10A, the implantable device 100 has a posterior side portion P (e.g., a first side portion), an anterior side portion A (e.g., a second side portion), a superior end portion S (e.g., a first end portion), and an inferior end portion I (e.g., a second end portion).

In the illustrated embodiment, the atrial-fixation member 1002 comprises a brim member 1050 at the anterior side portion A of the implantable device 1000 and arm members 1052 (including an individually identified first arm member 1052a and a second arm member 1052b) extending from the brim member 1050 to the coaptation member 1004. Referring to FIG. 10B, the first arm member 1052a extends between and couples a first side portion 1011 of the coaptation member 1004 to a first side portion 1051 of the brim member 1050, and the second arm member 1052b extends between and couples a second side portion 1013 of the coaptation member 1004 to a second side portion 1053 of the brim member 1050. In some aspects of the present technology, connecting the atrial-fixation member 1002 to the first and second side portion 1011, 1013 of the coaptation member 1004 (e.g., rather than an anterior portion 1012 of the coaptation member 1004) can increase the stability of the coaptation member 1004 when the implantable device 1000 is implanted at a native valve, thereby improving coaptation been coaptation member 1004 and one or more native leaflets of the native valve.

The brim member 1050 can have a generally curved shape in a circumferential direction about the flow axis VA selected to engage the tissue above a native valve (e.g., a native mitral valve) when the implantable device 1000 is implanted at the native valve. Moreover, in the illustrated embodiment the brim member 1050 extends only partially about the flow axis VA such that when the implantable device 1000 is implanted at the native valve, the brim member 1050 only engages a portion (e.g., an anterior portion) of the circumference of the native tissue above the valve (e.g., the tissue of the left atrium). For example, the brim member 1050 can have a length selected to extend between about 40°-100°, between about 45°-90°, and/or between about 50°-70° about the flow axis VA. In contrast, the atrial-fixation members 102, 202 shown in FIGS. 1A-9 extend entirely circumferentially (e.g., 360°) about the flow axis VA. In some aspects of the present technology, omitting a posterior portion of the atrial-fixation member 1002 can improve visualization of the implantable device 100 (e.g., the clip 1009) during delivery and implantation of the implantable device 1000. In some aspects of the present technology, the brim member 1050 provides fixation above the native valve (e.g., in the left atrium above the mitral valve) while the arm members 1052 help resist excess deflection of the coaptation member 1004.

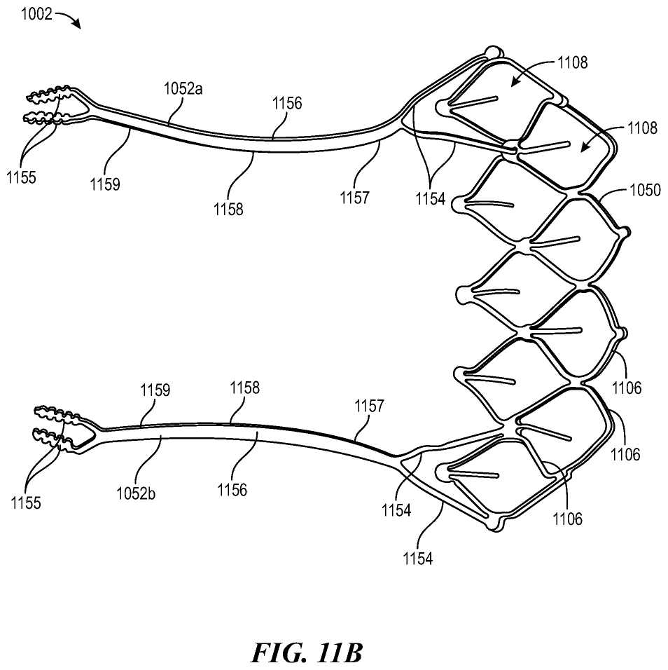

FIGS. 11A and 11B are a side view and a top view of the atrial-fixation member 1002 configured in accordance with embodiments of the present technology. Referring to FIGS. 11A and 11B, the atrial-fixation member 1002 can be formed of a mesh, such as a braid or laser-cut stent-like structure, including a plurality of interconnected wires or struts 1006 which together define a plurality of openings or cells 1008 (e.g., diamond-shaped openings) arranged in one or more rows (e.g., two rows). The struts 1006 can be configured to self-expand from a collapsed delivery state (not shown) to an expanded deployed state shown in FIGS. 11A and 11B. In some embodiments, the arm members 1152 are separate (e.g., formed from nitinol wire) from the brim member 1150 and attached thereto via suturing, welding, adhesives, fasteners, and/or the like. In other embodiments, the arm members 1152 are integrally formed with the brim member 1150. For example, the atrial-fixation member 1002 can be formed as a single laser cut structure.

In the illustrated embodiment, the arm members 1152 are identical or generally identical and each include a brim attachment portion 1154 and a coaptation attachment portion 1155. In the illustrated embodiment, the brim attachment portions 1154 and the coaptation attachment portions 1155 are each bifurcated or forked. The coaptation attachment portions 1155 are configured (e.g., shaped, sized, positioned) to be secured to the coaptation member 1004 (FIGS. 10A and 10B). For example, the coaptation attachment portions 1155 can be secured to the coaptation member 1004 (e.g., a stent, strut, and/or metal structure thereof) via suturing, welding, adhesives, fasteners, and/or the like. For example, with additional reference to FIG. 10B, the coaptation attachment portion 1155 (FIGS. 11A and 11B) of the first arm member 1052a is secured at and/or proximate to the first side portion 1011 of the coaptation member 1004, and the coaptation attachment portion 1155 (FIGS. 11A and 11B) of the second arm member 1052b is secured at and/or proximate to the second side portion 1013 of the coaptation member 1011. Likewise, the brim attachment portions 1154 can be secured to the brim member 1150 (e.g., the struts 1006) via suturing, welding, adhesives, fasteners, and/or the like, or integrally formed with the brim member 1150. In some embodiments, the brim attachment portions 1154 are arched or curved.

The arm members 1152 can each further include an arched portion 1156 extending between the brim attachment portion 1154 and the coaptation attachment portion 1155. In the illustrated embodiment, the arched portions 1156 each include (i) a generally straight anterior segment 1157 extending from the brim attachment portion 1154, (ii) an arched middle segment 1158 extending from the anterior segment 1157, and (iii) a generally straight posterior segment 1159 extending from the middle segment 1158 to the coaptation attachment portion 1155. In some embodiments, the middle segments can define an angle A (FIG. 11A) of between about 80°-120°, between about 50°-80°, and/or between about 60°-70°. Referring to FIGS. 10A-11B together, the arm members 1052 extend in an anterior direction away from the coaptation member 1004 to the brim member 1050 while, more specifically, (i) the posterior segments 1159 extend in the superior (e.g., upstream) direction away from the coaptation member 1004, (ii) the middle segments 1158 curvingly extend from the superior direction to the inferior (e.g., downstream) direction, and (iii) the anterior segments 1157 extend in the inferior direction to the brim member 1050. In the illustrated embodiment, the posterior segments 1159 are longer than the anterior segments 1157 such that the brim member 1050 is positioned superior to (e.g., upstream of) the coaptation member 1004.

Referring to FIGS. 10A and 10B, the implantable device 1000 can include one or more portions of fabric attached to the atrial-fixation member 1002 to facilitate long term tissue ingrowth into the structure of the atrial-fixation member 1002. For example, in the illustrated embodiment the implantable device 1000 includes a fabric member 1025 attached along a portion of the brim member 1050. The fabric member 1025 can comprise polyethylene terephthalate (PET) and/or another suitable material that facilitates long term tissue ingrowth, and can be secured to the brim member 1050 via sutures and/or other suitable fasteners. In some embodiments, the atrial-fixation member 1002 can include fixation features in the form of cleats. For example, the brim member 1050 can include upward facing anterior cleats 1015 (e.g., nine upward facing anterior cleats) configured to inhibit or even prevent migration of the implantable device 1000 into the left atrium.

In the illustrated embodiment, the coaptation member 1004 extends away from the arm members 1052 along the flow axis VA and at least a portion of the coaptation member 1004 extends radially inward from the arm members 1052 toward the flow axis VA to approximate a closed position of a native leaflet. The coaptation member 1004 can be substantially stationary (e.g., little to no movement) during cardiac cycles such that the position of the coaptation member 1004 relative to the atrial-fixation member 1002 is at least substantially fixed in the deployed state. Thus, unlike native leaflets that move back and forth to open and close the native valve, the coaptation member 1004 remains stationary during diastole and systole.

The coaptation member 1004 can have an anterior portion 1012 with a smooth, atraumatic surface for coapting with at least a portion of one or more native leaflets and a posterior portion 1014 configured to displace and, optionally, engage at least a portion of another native leaflet. The coaptation member 1004 can be made from a plurality of struts that form a basket-like or frame-like structure (e.g., a mesh structure, a laser cut stent frame) with an at least partially hollow interior and a covering (e.g., a fabric) extending over at least a portion of the struts to provide a smooth suitable surface for coaptation at the anterior portion 1012. The covering may also extend over the struts along the posterior portion 1014 and between the anterior and posterior portions 1012, 1014 in a manner that forms lateral sidewalls. The coaptation member 1004 or portions thereof can be integral with the arm members 1052 of the atrial-fixation member 1002 such that, for example, the coaptation member 1004 is manufactured from the same frame including the arm members 1052 and/or the brim member 1050. In other embodiments, the coaptation member 1004 can be a separate structure that is connected to the arm members 1052 during manufacturing. In some embodiments, the coaptation member 1004 can include a biocompatible foam which is attached to the structure of the coaptation member 1004 and/or to the arm members 1052. Referring to FIG. 10B, the covering can comprise a slit 1039 at a superior (e.g., top) portion of the coaptation member 1004 to allow for one or more components (e.g., a baffle screw, core plug, clip tendon) of an associated delivery system to attach to the coaptation member 1004. The slit 1039 can be configured to close after the coaptation member 1004 is detached from the associated delivery system.

In the illustrated embodiment, the coaptation member 1004 includes a brim portion 1060 (also referred to as a “secondary brim portion,” a “posterior brim,” or a “posterior extension”) that may be separate from and coupled to, or integral with, the coaptation member 1004. The brim portion 1060 extends away from a portion (e.g., a superior-posterior portion) of the coaptation member 1004 in a superior-posterior direction. The brim portion 1060 can be flexible. For example, the brim portion 1060 can comprise a nitinol wire form covered with PET fabric to provide a platform for long term tissue incorporation into the brim portion 1060. When the implantable device 1000 is implanted at a native valve, the brim portion 1060 can extend above the native valve annulus into the atrium (e.g., the left atrium) above the valve and can contact a portion (e.g., a posterior portion) of the native tissue above the valve (e.g., the tissue of the left atrium). In some aspects of the present technology, the brim portion 1060 is relatively small compared to, for example, the posterior portion of the atrial-fixation members 102, 202 shown in FIGS. 1A-9. This can help minimize undesirable interaction of the implantable device 1000 with the left atrium that may otherwise move or shift the implantable device 1000 relative to the native valve.

Referring to FIG. 10A, the clip 1009 depends from the posterior portion 1014 of the coaptation member 1004 and can be opened to extend behind the native leaflet the coaptation member 1004 displaces. The clip 1009 may grasp the native leaflet and/or engage sub-annular cardiac tissue for sub-annular stabilization of the implantable device 1000. In some embodiments, for example, the clip 1009 reaches under the central portion (i.e., P2) of the posterior leaflet up to the sub-annular space. A tendon (made of suture or nitinol wire) can actuate the clip 1009 by way of a lever attached to the clip 1009. The lever may be a nitinol wire or laser cut nitinol or Co-Cr sheet.

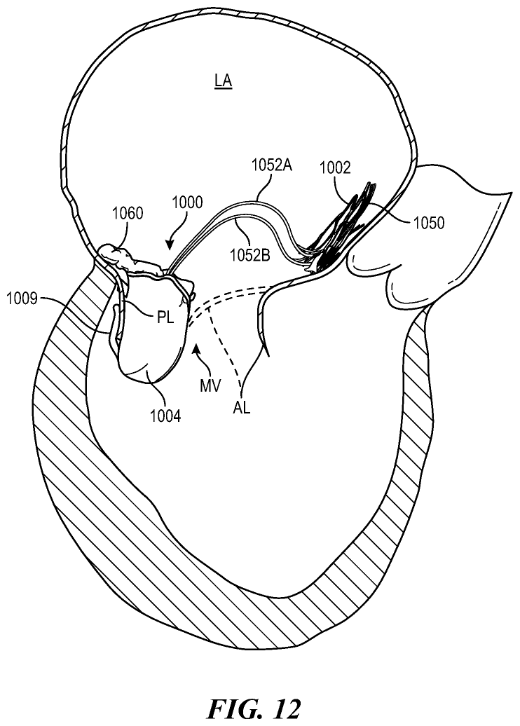

FIG. 12 is a side cross-sectional view of the implantable device 1000 of FIGS. 10A-11B implanted at a mitral valve in accordance with embodiments of the present technology. In the illustrated embodiment, when the implantable device 1000 is deployed across the mitral valve annulus, the coaptation member 1004 may extend in front of a portion (e.g., a central portion) of a posterior leaflet PL (e.g., the P2 scallop of the posterior leaflet PL) to position the coaptation member 1004 in a location that allows it to coapt with an anterior leaflet AL during systole (the anterior leaflet is shown during systole in broken lines and in diastole in solid lines in FIG. 12). The clip 1009 is configured to extend behind and grasp portions of one or more native leaflets to affix the one or more leaflets to the coaptation member 1004. The brim member 1050 contacts an anterior portion of the left atrium LA, and the brim portion 1060 of the coaptation member 1004 extends above the posterior leaflet PL into the left atrium LA. The arm members 1052 extend in an arch into the left atrium LA and to the brim member 1050 to brace and fix the coaptation member 1004 in position.

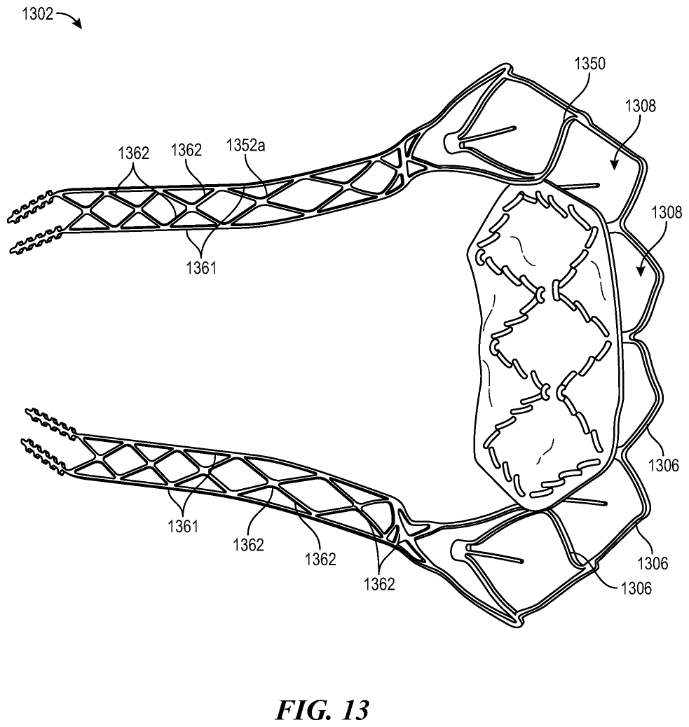

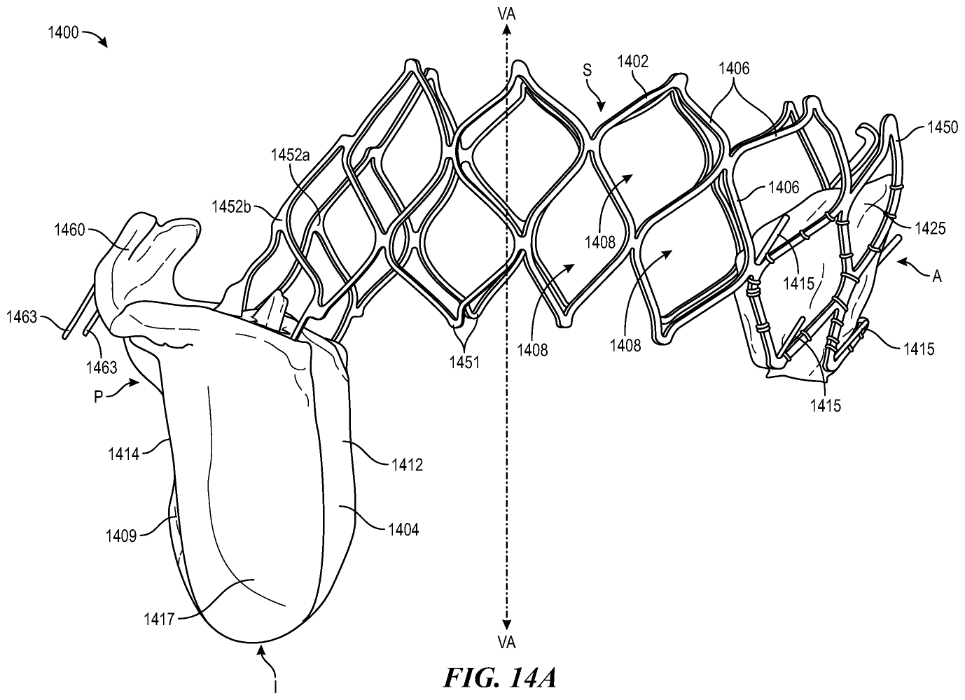

FIG. 13 is a top view of an atrial-fixation member 1302 configured in accordance with additional embodiments of the present technology. The atrial-fixation member 1002 can include some features that are at least generally similar in structure and function, or identical in structure and function, to the corresponding features of the atrial-fixation member 1002 described in detail above with reference to FIGS. 10A-11B, and can operate in a generally similar or identical manner to the atrial-fixation member 1002 shown in FIGS. 10A-11B. For example, the atrial-fixation member 1302 can be incorporated into the implantable device 1000 instead of the atrial-fixation member 1002.

In the illustrated embodiment, the atrial-fixation member 1302 comprises a brim member 1350 and arm members 1352 (including an individually identified first arm member 1352a and a second arm member 1352b) extending from the brim member 1350 and configured to be coupled to a coaptation member (e.g., the coaptation member 1004 of FIGS. 10A and 10B). The first arm member 1352a extends between and couples a first side portion of the coaptation member to a first side portion of the brim member 1350, and the second arm member 1352b extends between and couples a second side portion of the coaptation member to a second side portion of the brim member 1350. The brim member 1350 can have a generally curved shape in a circumferential direction about a flow axis of the implantable device selected to engage the tissue above a native valve (e.g., a native mitral valve) when the implantable device is implanted at the native valve. Moreover, in the illustrated embodiment the brim member 1350 extends only partially about the flow axis such that when the implantable device is implanted at the native valve, the brim member 1350 only engages a portion (e.g., an anterior portion) of the circumference of the native tissue above the valve (e.g., the tissue of the left atrium). The atrial-fixation member 1302 can be formed of a mesh, such as a braid or laser-cut stent-like structure, including a plurality of interconnected wires or struts 1306 which together define a plurality of openings or cells 1308 (e.g., diamond-shaped openings) arranged in one or more rows (e.g., two rows). In the illustrated embodiment, the arm members 1352 each comprise a pair of generally parallel first (e.g., outer) struts 1361 connected by second (e.g., inner) struts 1362. The second struts 1362 can have a diamond like shape.

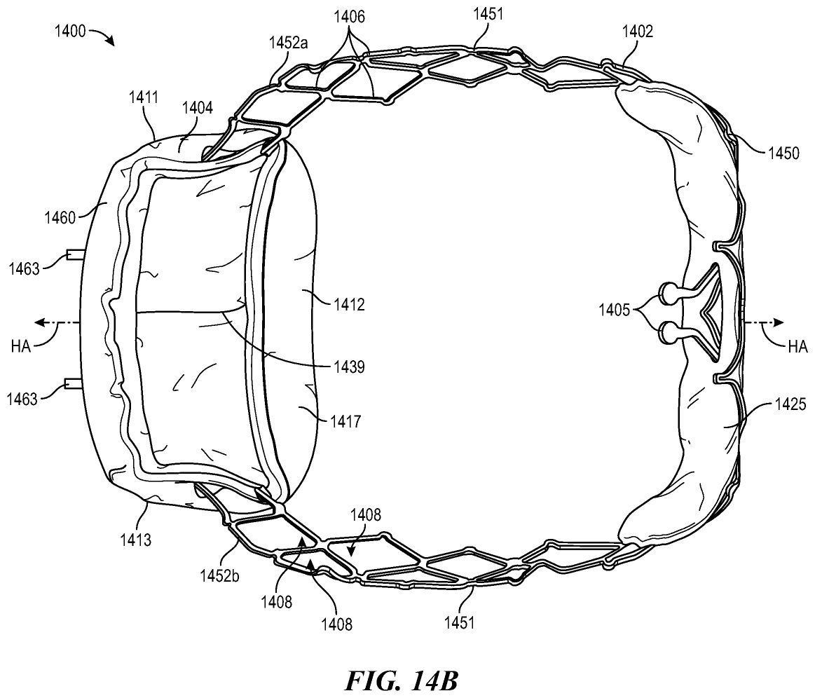

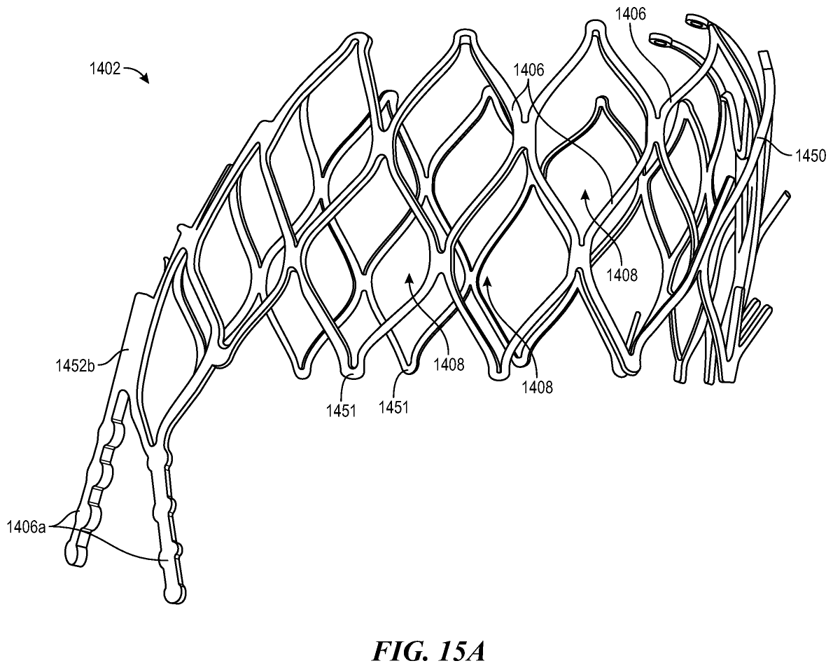

FIGS. 14A and 14B are a side view (e.g., an anterior-posterior side view) and a top view (e.g., an en face view) of an implantable device 1400 that can be implanted within a heart of a subject (e.g., a human patient) in accordance with embodiments of the present technology. The implantable device 1400 can include some features that are at least generally similar in structure and function, or identical in structure and function, to the corresponding features of the implantable device 100, the implantable device 200, and/or the implantable device 1000 described in detail above with reference to FIGS. 1A-13, and can operate in a generally similar or identical manner to the implantable devices shown in FIGS. 1A-13.

For example, referring to FIGS. 14A and 14B, in the illustrated embodiment the implantable device 1400 is a valve repair device having an atrial-fixation member 1402 (also referred to as an “anterior brim,” an “anterior-only brim,” an “anterior anchoring member,” or a “suprannular anchoring member”) and a coaptation member 1404 extending from the atrial-fixation member 1402 in a downstream direction. The atrial-fixation member 1402 is configured to anchor the implantable device 1400 to cardiac tissue proximate to a native mitral valve annulus and position the coaptation member 1404 at a desired location with respect to the native valve anatomy of the heart. The implantable device 1400 is designed to eliminate regurgitation by restoring physiologic coaptation in a diseased mitral valve. The coaptation member 1404 can be covered in a biocompatible material (e.g., polytetrafluoroethylene (PTFE), expanded polytetrafluoroethylene (ePTFE)) and shaped (e.g., contoured) to fill a regurgitant orifice of the mitral valve from the posterior side and provide a new coaptation surface for the native leaflets of the mitral valve. The atrial-fixation member 1402 can be flexible and can provide additional fixation and supra-annular stabilization. In the illustrated embodiment, the implantable device 1400 further includes a posterior clip 1409 (obscured in FIG. 14B) that can orient the implantable device 1400 and provide sub-annular fixation.

The implantable device 1400 is configured relative to a flow axis VA (FIG. 14A) in the direction of blood flow from the atrium to the ventricle and a transverse axis HA (FIG. 14B) at an angle (e.g., orthogonal) to the flow axis VA. As shown in FIG. 14A, the implantable device 1400 has a posterior side portion P (e.g., a first side portion), an anterior side portion A (e.g., a second side portion), a superior end portion S (e.g., a first end portion), and an inferior end portion I (e.g., a second end portion).

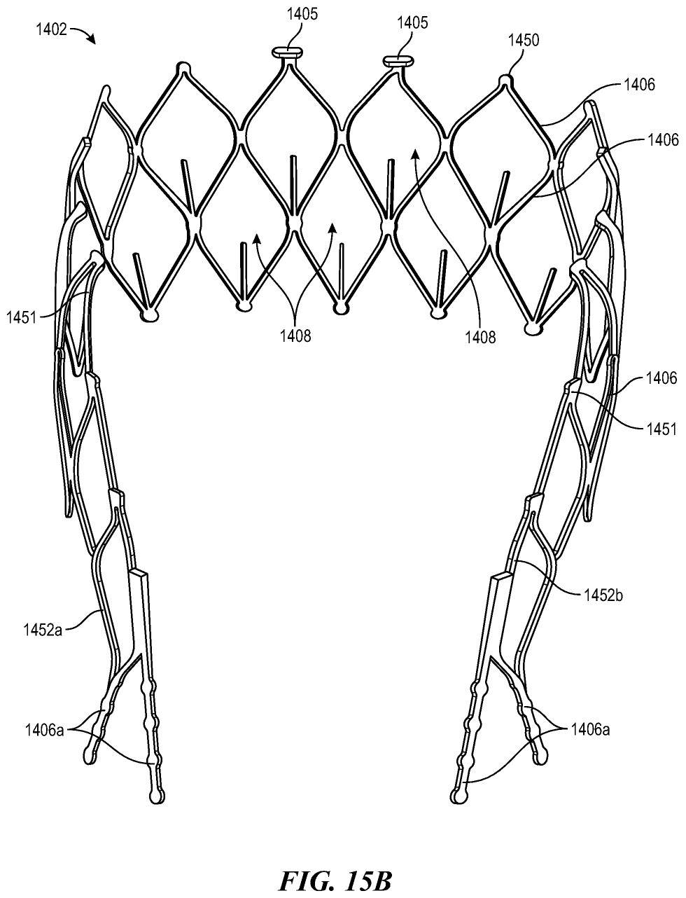

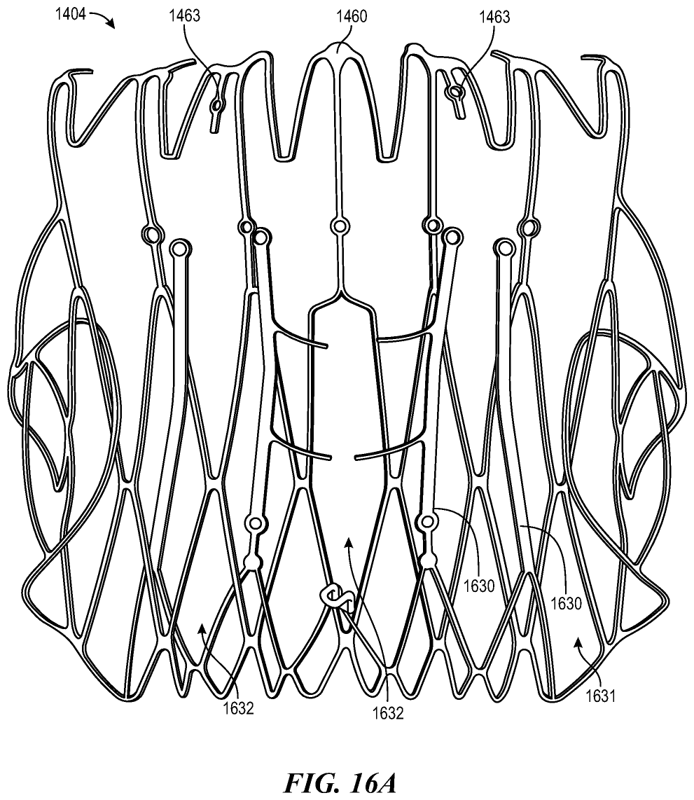

FIGS. 15A and 15B are a side view and a perspective top view of the atrial-fixation member 1402 configured in accordance with embodiments of the present technology. Referring to FIGS. 14A-15B, the atrial-fixation member 1402 can be formed of a mesh, such as a braid or laser-cut stent-like structure, including a plurality of interconnected wires or struts 1406 which together define a plurality of openings or cells 1408 (e.g., diamond-shaped openings) arranged in one or more rows (e.g., two rows). The struts 1406 can be configured to self-expand from a collapsed delivery state (not shown) to an expanded deployed state shown in FIGS. 14A and 14B. In the illustrated embodiment, the atrial-fixation member 1402 comprises (i) an anterior brim portion 1450 at and/or proximate the anterior side portion A of the implantable device 1400, (ii) a pair of medial extension portions 1451 extending in a posterior direction from the brim portion 1450, and (iii) a pair of posterior connection portions 1452 (including an individually identified first posterior connection portion 1452a and a second posterior connection portion 1452b) extending in a posterior-inferior direction from the extension portions 1451 to the coaptation member 1404. More specifically, as shown in FIG. 14B, the first posterior connection portion 1452a is secured at and/or proximate to a first side portion 1451 of the coaptation member 1404, and the second posterior connection portion 1452b is secured at and/or proximate to a first side portion 1413 of the coaptation member 1404. In some aspects of the present technology, connecting the atrial-fixation member 1402 to the first and second side portion 1411, 1413 of the coaptation member 1404 (e.g., rather than an anterior portion 1412 of the coaptation member 1404) can increase the stability of the coaptation member 1404 when the implantable device 1400 is implanted at a native valve, thereby improving coaptation been coaptation member 1404 and one or more native leaflets of the native valve.

The brim portion 1450 can have a generally curved shape in a circumferential direction about the flow axis VA selected to engage the tissue above a native valve (e.g., a native mitral valve) when the implantable device 1400 is implanted at the native valve. Moreover, in the illustrated embodiment the brim portion 1450 extends only partially about the flow axis VA such that when the implantable device 1400 is implanted at the native valve, the brim portion 1450 only engages a portion (e.g., an anterior portion) of the circumference of the native tissue above the valve (e.g., the tissue of the left atrium). For example, the brim portion 1450 can have a length selected to extend only between about 40°-140°, between about 45°-90°, and/or between about 50°-70° about the flow axis VA. In contrast, the atrial-fixation members 102, 202 shown in FIGS. 1A-9 extend entirely circumferentially (e.g., 360°) about the flow axis VA. In some aspects of the present technology, omitting a posterior portion of the atrial-fixation member 1402 can improve visualization of the implantable device 1400 (e.g., the clip 1409) during delivery and implantation of the implantable device 1400. In some aspects of the present technology, the atrial-fixation member 1402 provides fixation above the native valve (e.g., in the left atrium above the mitral valve) and also helps resist excess deflection of the coaptation member 1404.

As best seen in FIGS. 14B and 15B, the atrial-fixation member 1402 can have a circumferential U-like shape about the flow axis VA (FIG. 14A). In some embodiments, the extension portions 1451 taper inward toward the flow axis VA in the anterior direction from the brim portion 1450 to the coaptation member 1404. Referring to FIGS. 15A and 15B, one or more of the struts 1406 (identified as “attachment struts 1406a”) near a posterior end of each of the connection portions 1452 can define a region for attachment to the coaptation member 1404 (FIGS. 14A and 14B). The attachment struts 1406a are configured (e.g., shaped, sized, positioned) to be secured to the coaptation member 1404 (e.g., a stent, strut, and/or metal structure thereof) via suturing, welding, adhesives, fasteners, and/or the like. Referring to FIGS. 14A and 14B, the connection portions 1452 act to position the brim portion 1450 and the extension portions 1451 generally superior to (e.g., upstream of) the coaptation member 1404 (e.g., above the anterior portion 1412 and coaptation surface of the coaptation member 1404). Referring to FIG. 14A, in some embodiments the atrial-fixation member 1402 can curve or taper downward (e.g., in the inferior direction) in the anterior direction. For example, the atrial-fixation member 1402 can have a curved or “rainbow” shape in which (i) an uppermost portion of the connection portions 1452 is positioned above an uppermost portion of the extension portions 1451, and (ii) an uppermost portion of the extension portions 1451 is positioned above an uppermost portion of the brim portion 1450. That is, a superior portion of the atrial-fixation member 1402 can taper downward relative to the flow axis VA from the extension portions 1451 toward the brim portion 1450. In some aspects of the present technology, such a curved shape of the atrial-fixation member 1402 can help inhibit interference of the atrial-fixation member 1402 with the medial and lateral aspects of native leaflets when the implantable device is 1400 is positioned at a native valve. FIGS. 15A and 15B illustrate the atrial-fixation member 1402 without such a curved or “rainbow” shape and instead including a superior portion having a generally linear or flat shape relative to the transverse axis HA (FIG. 10B).

In some embodiments, the implantable device 1400 can include one or more portions of fabric attached to the atrial-fixation member 1402 to facilitate long term tissue ingrowth into the structure of the atrial-fixation member 1402. For example, in the illustrated embodiment the implantable device 1400 includes a fabric member 1425 attached along a portion of the brim portion 1450. The fabric member 1425 can comprise polyethylene terephthalate (PET) and/or another suitable material that facilitates long term tissue ingrowth, and can be secured to the brim portion 1450 via sutures and/or other suitable fasteners (e.g., adhesives). In some embodiments, the atrial-fixation member 1402 can include fixation features in the form of cleats. For example, the brim portion 1450 can include upward facing anterior cleats 1415 (e.g., nine upward facing anterior cleats) and/or downward facing posterior cleats 1463 (e.g., two downward facing posterior cleats) to inhibit or even prevent migration of the implantable device 1400 into the left atrium. In some embodiments, the downward facing posterior cleats 1463 originate from (e.g., extend from) the coaptation member 204. As best seen in FIGS. 14B and 15B, the atrial-fixation member 1402 can include connectors 1405 that are configured (e.g., sized, shaped, and/or positioned) to engage with one or more mating features on an associated delivery system. The connectors 1405 can extend from the struts 1406 such that the connectors 1405 are positioned at or near the superior end portion S (FIG. 14A) of the implantable device 1400. In the illustrated embodiment, the implantable device 1400 includes two of the connectors 1405. In some aspects of the present technology, the connectors 1405 can help maintain the implantable device 1400 in a rotatably stable position as the clip 1409 is opened and closed.

In the illustrated embodiment, the coaptation member 1404 extends away from the connection portions 1452 along the flow axis VA and at least a portion of the coaptation member 1404 extends radially inward from the connection portions 1452 toward the flow axis VA to approximate a closed position of a native leaflet. The coaptation member 1404 can be substantially stationary (e.g., little to no movement) during cardiac cycles such that the position of the coaptation member 1404 relative to the atrial-fixation member 1402 is at least substantially fixed in the deployed state. Thus, unlike native leaflets that move back and forth to open and close the native valve, the coaptation member 1404 remains stationary during diastole and systole.

The coaptation member 1404 or portions thereof can be integral with the connection portions 1452 of the atrial-fixation member 1402 such that, for example, the coaptation member 1404 is manufactured from the same frame including the atrial-fixation member 1402. In other embodiments, the coaptation member 1404 can be a separate structure that is connected to connection portions 1452 during manufacturing. In some embodiments, the coaptation member 1404 can include a biocompatible foam which is attached to the structure of the coaptation member 1404 and/or to the atrial-fixation member 1402.

The coaptation member 1404 can have an anterior portion 1412 with a smooth, atraumatic surface for coapting with at least a portion of one or more native leaflets and a posterior portion 1414 configured to displace and, optionally, engage at least a portion of another native leaflet. In the illustrated embodiment, the coaptation member 1404 includes a brim member 1460 (also referred to as a “posterior brim” or a “posterior extension”) that may be separate from and coupled to, or integral with the coaptation member 1404. The brim member 1460 extends away from a portion (e.g., a superior-posterior portion) of the coaptation member 1404 in a superior-posterior direction. The brim member 1460 can be flexible. For example, the brim member 1460 can comprise a nitinol wire form covered with PET fabric to provide a platform for long term tissue incorporation into the brim member 1460. When the implantable device 1400 is implanted at a native valve, the brim member 1460 can extend above the native valve annulus into the atrium (e.g., the left atrium) above the valve and can contact a portion (e.g., a posterior portion) of the native tissue above the valve (e.g., the tissue of the left atrium). In some aspects of the present technology, the brim member 1460 is relatively small compared to, for example, the posterior portion of the atrial-fixation members 142, 202 shown in FIGS. 1A-9. This can help minimize undesirable interaction of the implantable device 1400 with the left atrium that may otherwise move or shift the implantable device relative to the native valve.

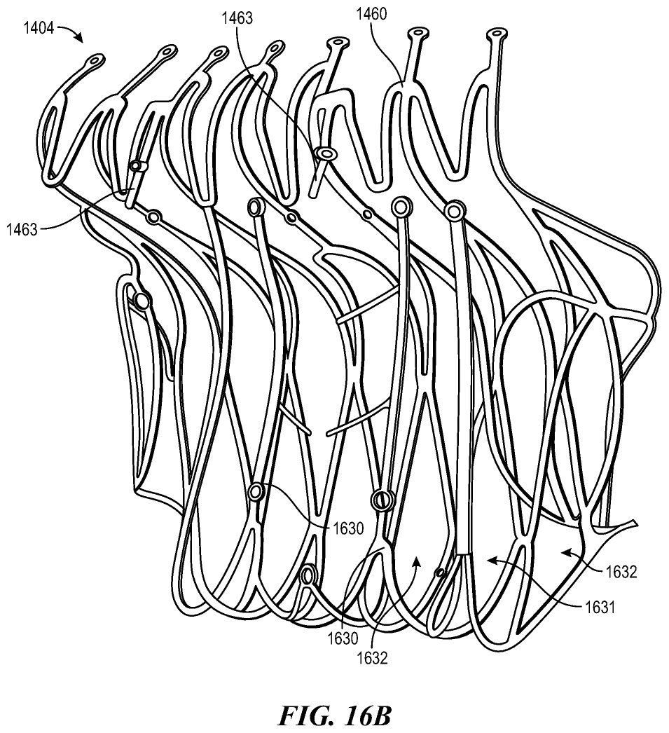

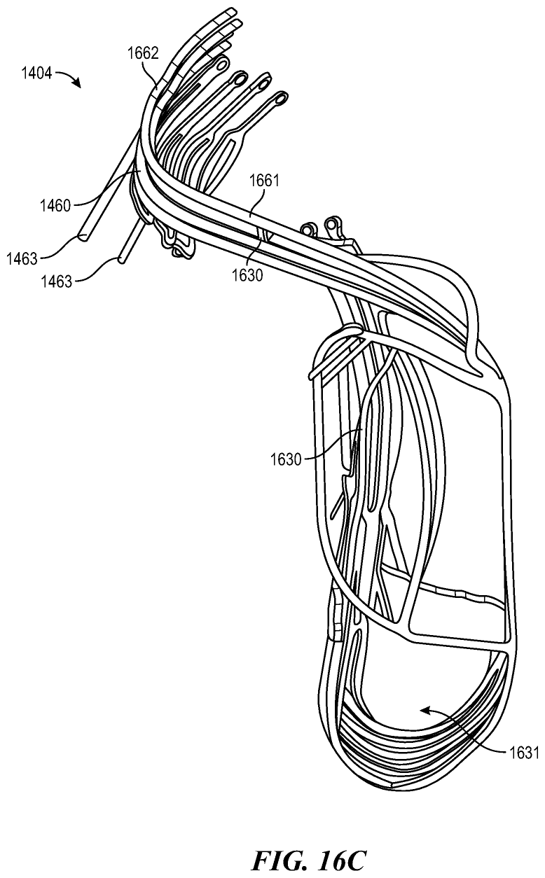

The coaptation member 1404 can be made from a plurality of struts that form a basket-like or frame-like structure (e.g., a mesh structure, a laser cut stent frame) with an at least partially hollow interior and a covering 1417 (e.g., a fabric) extending over at least a portion of the struts to provide a smooth suitable surface for coaptation at the anterior portion 1412. More specifically, FIGS. 16A-16C are side views (e.g., an anterior side view, a perspective posterior side view, and an anterior-posterior side view, respectively) of the coaptation member 104 with the covering 1417 removed in accordance with embodiments of the present technology. The coaptation member 1404 can be formed of a mesh, such as a braid or laser-cut stent-like structure, including a plurality of interconnected wires or struts 1630 which together define a plurality of openings or cells 1632 (partially obscured in FIG. 16C). The struts 1630 further define a hollow interior 1631 and the brim member 1460. As best seen in FIG. 16C, the struts 1630 at the brim member 1460 can each include (i) a generally straight segment 1661 extending from the struts 1630 surrounding the hollow interior 1631 and (ii) an arched or curved superior segment 1662 extending from the generally straight segment 1661. The straight segments 1661 can extend in a posterior-superior direction, and the superior segments 1662 can extend in the superior direction while also curving from the posterior direction to the anterior direction. In some embodiments, the posterior cleats 1463 can be integral with and/or formed by the struts 1630 and can extend in the posterior-inferior (e.g., downward) direction from corresponding ones of the superior segments 1662.

With additional reference to FIGS. 14A and 14B, the covering 1417 of the coaptation member 1404 and the brim member 1460 can extend over the struts 1630 along the posterior portion 1414 and between the anterior and posterior portions 1412, 1414 in a manner that forms lateral sidewalls. Referring to FIG. 14B, the covering 1417 can comprise a slit 1439 at a superior (e.g., top) portion of the coaptation member 1404 to allow for one or more components (e.g., a baffle screw, core plug, clip tendon) of an associated delivery system to attach to the coaptation member 1404. The slit 1439 can be configured to close after the coaptation member 1404 is detached from the associated delivery system.

Referring to FIG. 14A, the clip 1409 depends from the posterior portion 1414 of the coaptation member 1404 and can be opened to extend behind the native leaflet the coaptation member 1404 displaces. The clip 1409 may grasp the native leaflet and/or engage sub-annular cardiac tissue for sub-annular stabilization of the implantable device 1400. In some embodiments, for example, the clip 1409 reaches under the central portion (i.e., P2) of the posterior leaflet up to the sub-annular space. A tendon (made of suture or nitinol wire) can actuate the clip 1409 by way of a lever attached to the clip 1409. The lever may be a nitinol wire or laser cut nitinol or Co-Cr sheet.

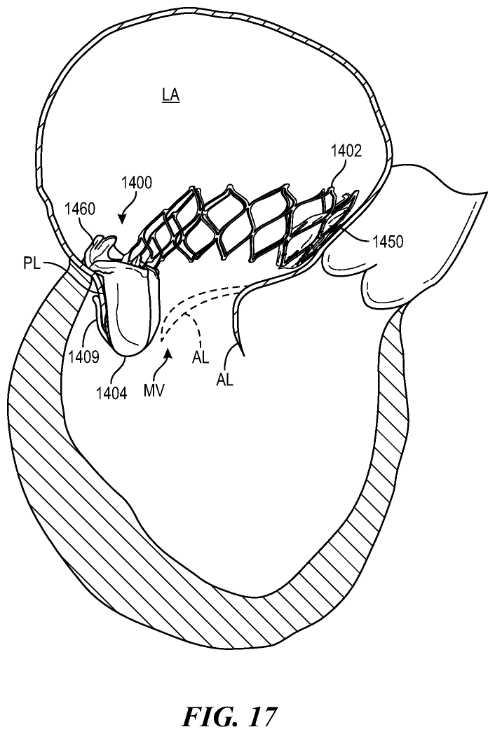

FIG. 17 is a side cross-sectional view of the implantable device 1400 of FIGS. 14A-16C implanted at a mitral valve MV in accordance with embodiments of the present technology. In the illustrated embodiment, when the implantable device 1400 is deployed across the mitral valve MV annulus, the coaptation member 1404 may extend in front of a portion (e.g., a central portion) of a posterior leaflet PL (e.g., the P2 scallop of the posterior leaflet PL) to position the coaptation member 1404 in a location that allows it to coapt with an anterior leaflet AL during systole (the anterior leaflet is shown during systole in broken lines and in diastole in solid lines in FIG. 17). The clip 1409 is configured to extend behind and grasp portions of one or more native leaflets to affix the one or more leaflets to the coaptation member 1404. The brim portion 1450 of the atrial-fixation member 1402 contacts an anterior portion of the left atrium LA, and the brim member 1460 of the coaptation member 1404 extends above the posterior leaflet PL into the left atrium. The atrial-fixation member 1402 extends in an anterior-superior direction from the coaptation member 1404 into the left atrium LA to brace and fix the coaptation member 1404 in position.

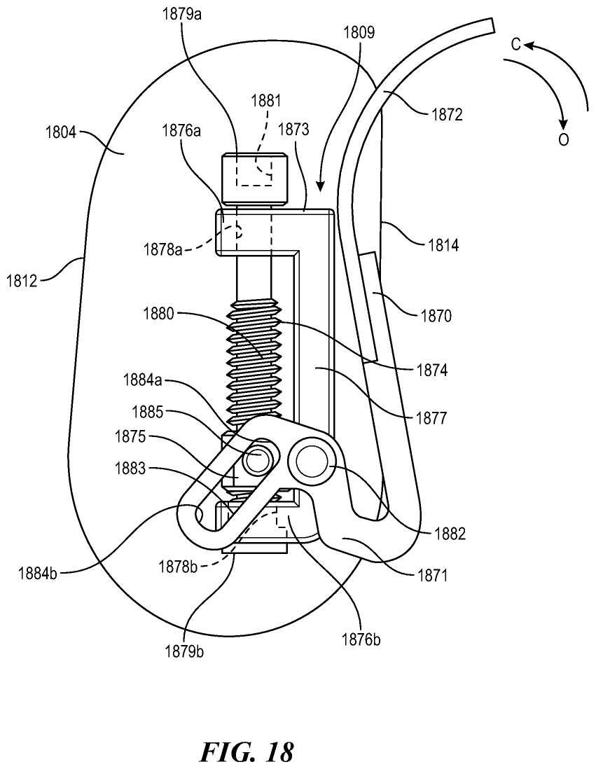

FIGS. 18-22B illustrate additional embodiments of clip assemblies (also referred to as “clips,” “clip mechanisms,” and the like) for use with the implantable devices of the present technology. Accordingly, any of the clip assemblies and/or components thereof described in detail with reference to FIGS. 18-22B can be incorporated into any of the implantable devices described in detail with reference to FIGS. 1A-17.

FIG. 18 is a side view of a coaptation member 1804 and an associated clip assembly 1809 configured in accordance with embodiments of the present technology. The coaptation member 1804 is shown as partially transparent in FIG. 18 for clarity. The coaptation member 1804 is configured to be secured to an atrial-fixation member (not shown) and can have an anterior portion 1812 with a smooth, atraumatic surface for coapting with at least a portion of one or more native leaflets and a posterior portion 1814 configured to displace and, optionally, engage at least a portion of another native leaflet. In the illustrated embodiment, the clip assembly 1809 comprises (i) a clip member 1870 having a root portion 1871 and an arm portion 1872 extending from the root portion 1871, (ii) a back member 1873 positioned within the coaptation member 1804, (iii) a threaded member 1874, and (iv) an actuation member 1875. Some or all of the components of the clip assembly 1809 can be made from cobalt-chromium (Co-Cr), nitinol, stainless steel (e.g., SS316L stainless steel), and/or the like, and can be machined via, for example, wire electrical discharge machining (WEDM).

The back member 1873 can be secured to an internal structure (e.g., a stent structure) of the coaptation member 1804 such that it is fixedly attached to the coaptation member 1804. The clip assembly 1809 is shown in a closed configuration (e.g., a closed position) in FIG. 18 in which the arm portion 1872 of the clip member 1870 abuts and/or is proximate to the coaptation member 1804 (e.g., the posterior portion 1814 of the coaptation member 1804). In some embodiments, in the closed configuration, the arm portion 1872 can be at least partially positioned in a recess in the covering of the coaptation member 1804.

In the illustrated embodiment, the back member 1873 has a first (e.g., upper) horizontal portion 1876a and a second (e.g., lower) horizontal portion 1876b connected to respective end portions of an elongate vertical portion 1877. The first horizontal portion 1876a defines a first aperture 1878a therethrough, and the second horizontal portion 1876b defines a second aperture 1878b therethrough. In some embodiments, the second aperture 1878b can be stepped. In the illustrated embodiment, the threaded member 1874 can have a first (e.g., upper) head 1879a and a second (e.g., lower) head 1879b connected by a partially threaded rod 1880. The rod 1880 can extend through the first aperture 1878a and partially through the second aperture 1878b and can rotate therein. In some embodiments, the second head 1879b is rotatably retained within the stepped second aperture 1878b such that the second head 1879b is prevented from moving upward through the second aperture 1878b. Similar, the first head 1879a is positioned above the first horizontal portion 1876a and sized such that the first head 1879a cannot pass downward through the first aperture 1878a. The first head 1879a can have a drive recess 1881 (e.g., a slotted recess, a hex recess, a square recess, and/or the like) for releasably receiving a component of an associated delivery system. The component of the associated delivery system can be actuated (e.g., rotated) to rotate the threaded member 1874.

In the illustrated embodiment, the root portion 1871 is pivotably coupled to the vertical portion 1877 of the back member 1873 via, for example, a pin 1882. The root portion 1871 further includes a slot 1883 having a first end portion 1884a and a second end portion 1884b. The slot 1883 can extend generally linearly and, in the closed configuration shown in FIG. 18, is angled off vertical (e.g., angled relative to the threaded member 1874 and/or the vertical portion 1877 of the back member 1873). The actuation member 1875 can be a threaded nut secured to the threaded portion of the rod 1880. In the illustrated embodiment, the actuation member 1875 includes a projection 1885 extending into the slot 1883 of the root portion 1871.

In the closed configuration shown in FIG. 18, the projection 1885 is positioned at or proximate the first end portion 1884a of the slot 1883. To actuate the clip assembly 1809 to open the arm portion 1872—e.g., pivot the arm portion 1872 away from the coaptation member 1804 in the direction of arrow 0—the threaded member 1874 can be rotated in a first direction via the component of the associated delivery system which engages the drive recess 1881. Rotation of the threaded member 1874 in the first direction drives the actuation member 1875 upward toward the first horizontal portion 1876a along the threads of the rod 1880. As the actuation member 1875 moves upward, the projection 1885 moves along the slot 1883 from the first end portion 1884a toward the second end portion 1884b and drives the root portion 1871 to pivot about the pin 1882 in a clockwise direction and thus the arm portion 1872 to pivot open in the direction of the arrow 0. The clip assembly 1809 can reach a fully open position when the projection 1885 reaches the second end portion 1884b of the slot 1883. In some embodiments, the arm portion 1872 can pivot open by about 90° relative to the back member 1873 and the posterior portion 1814 of the coaptation member 1804. The second end portion 1884b can function as a hard stop that prevents further opening of the clip member 1870.

To actuate the clip assembly 1809 to close the arm portion 1872—e.g., pivot the arm portion 1872 toward the coaptation member 1804 in the direction of arrow C—the threaded member 1874 can be rotated in a second direction opposite the first direction via the component of the associated delivery system which engages the drive recess 1881. Rotation of the threaded member 1874 in the second direction drives the actuation member 1875 downward toward the second horizontal portion 1876b along the threads of the rod 1880. As the actuation member 1875 moves downward, the projection 1885 moves along the slot 1883 from the second end portion 1884b toward the first end portion 1884a and drives the root portion 1871 to pivot about the pin 1882 in a counterclockwise direction and thus the arm portion 1872 to pivot closed in the direction of the arrow C.

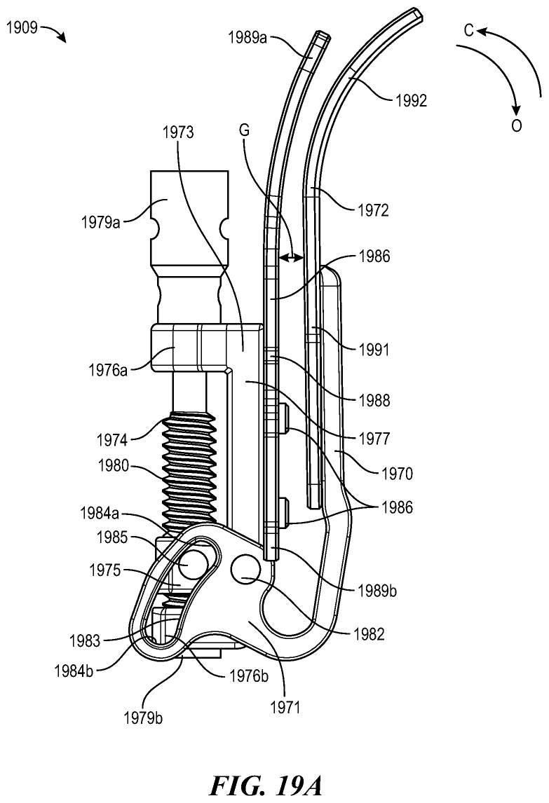

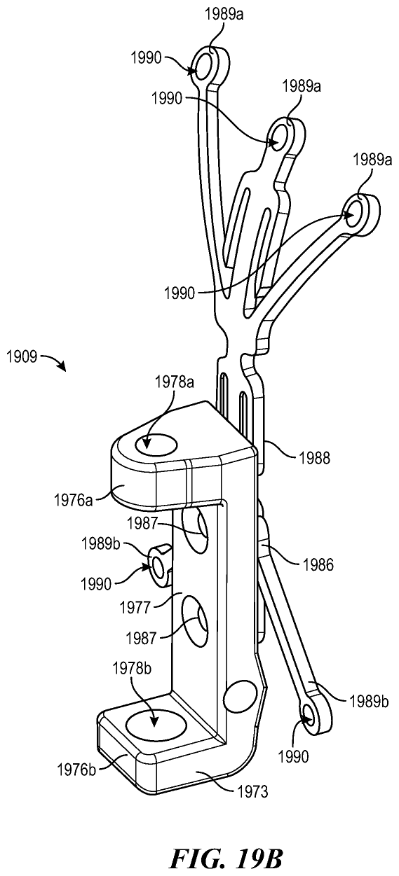

FIG. 19A is a side view of a clip assembly 1909 configured in accordance with additional embodiments of the present technology. The clip assembly 1909 can include some features that are at least generally similar in structure and function, or identical in structure and function, to the corresponding features of the clip assembly 1809 described in detail above with reference to FIG. 18, and can operate in a generally similar or identical manner to the clip assembly shown in FIG. 18.

For example, in the illustrated embodiment the clip assembly 1909 comprises (i) a clip member 1970 having a root portion 1971 and an arm portion 1972 extending from the root portion 1971, (ii) a back member 1973 configured to be positioned within a coaptation member, (iii) a threaded member 1974, and (iv) an actuation member 1975. The back member 1973 has a first (e.g., upper) horizontal portion 1976a and a second (e.g., lower) horizontal portion 1976b connected to respective end portions of an elongate vertical portion 1977 and having first and second apertures 1978a-b (FIG. 19B), respectively, extending vertically therethrough. In the illustrated embodiment, the threaded member 1974 has (i) a first (e.g., upper) head 1979a positioned above the first horizontal portion 1976a, (ii) a second (e.g., lower) head 1979b rotatably retained in the second aperture 1978b (FIG. 19B) in the second horizontal portion 1976b, and (iii) a partially threaded rod 1980 extending between the upper and lower heads 1979a-b and at least partially through the first and second apertures 1978a-b (FIG. 19B) in the first and second horizontal portions 1976a-b, respectively. The first head 1979a can have a driving recess (e.g., a slotted recess, a hex recess, a square recess, and/or the like) for releasably receiving a component of an associated delivery system. The root portion 1971 is pivotably coupled to the vertical portion 1977 of the back member 1973 via, for example, a pin 1982. The root portion 1971 further includes a slot 1983 having a first end portion 1984a and a second end portion 1984b. In some embodiments, the slot 1983 can have a V-shape that is, in the closed configuration shown in FIG. 19A, angled off vertical (e.g., angled downward relative to the threaded member 1974 and/or the vertical portion 1977 of the back member 1973). The actuation member 1975 can be a threaded nut secured to the threaded portion of the rod 1980 and having a projection 1985 extending into the slot 1983 of the root portion 1971.

In the illustrated embodiment, the clip assembly 1909 further includes an attachment member 1986 (also referred to as a “spider member”) coupled to the vertical portion 1977 of the back member 1973 opposite the threaded member 1974. For example, the attachment member 1986 can be secured via rivets 1987 to the back member 1973. FIG. 19B is a perspective side view of the clip assembly 1909 showing only the back member 1973 and the attachment member 1986 coupled thereto in accordance with embodiments of the present technology. Referring to FIGS. 19A and 19B, the attachment member 1986 can include (i) a middle body portion 1988, (ii) first (e.g., upper) arm or finger portions 1989a (e.g., three finger portions 1989a) extending upward from the body portion 1988, and (iii) second (e.g., lower) arm or finger portions 1989b (e.g., three finger portions 1989b) extending downward from the body portion 1988. The first and second finger portions 1989a-b can splay outward from the body portion 1988. The body portion 1988 and/or one or more of the second finger portions 1989b can be secured to the back member 1973 via the rivets 1987.

The attachment member 1986 is configured to be secured to a coaptation member of an implantable device such that the clip assembly 1909 is fixedly attached to the coaptation member. In some embodiments, the attachment member 1986 can be secured to the coaptation member via suturing. For example, one or more sutures can be threaded through eyelets or apertures 1990 (FIG. 19B) formed in distal end portions of one or more of the first and second finger portions 1989a-b. In some aspects of the present technology, securing the attachment member 1986 to the coaptation member via suturing can reduce the likelihood of corrosion as compared to, for example, fastening techniques that require metal interconnections (e.g., riveting).

In the illustrated embodiment, the attachment member 1986 has a planform shape (e.g., as shown in the side view of FIG. 19A) that generally tracks or matches the planform shape of the arm portion 1972 of the clip member 1970. For example, the arm portion 1972 can have a generally flat first (e.g., lower) segment 1991 coupled to the root portion 1971 and a curved second (e.g., upper) segment 1992 extending from the first segment 1991. The second segment 1992 can curve in the posterior direction away from, for example, the back member 1973. Similarly, the body portion 1988 and the second finger portions 1989b of the attachment member 1986 can be generally flat, and the first finger portions 1989a of the attachment member 1986 can be curved. In the closed configuration shown in FIG. 19A, the body portion 1988 and the second finger portions 1989b of the attachment member 1986 can be positioned adjacent to and generally parallel to the first segment 1991 of the arm portion 1972, and the first finger portions 1989a of the attachment member 1986 can be positioned adjacent to and generally parallel to the second segment 1992 of the arm portion 1972. Accordingly, in the closed configuration, the arm portion 1972 can be spaced apart from the attachment member 1986 by a gap G having a generally constant dimension (e.g., within about 20% of a set value along the length of the arm portion 1972). In some aspects of the present technology, this configuration of the arm portion 1972 and the attachment member 1986 allows the clip assembly 1909 to clamp a leaflet positioned between the attachment member 1986 and the arm portion 1972 along substantially the entire length of the arm portion 1972. This can improve the fixation of the leaflet within the clip assembly 1909.

Referring to FIG. 19A, in the illustrated closed configuration, the projection 1985 is positioned at or proximate the first end portion 1984a of the slot 1983, which is angled off vertical. To actuate the clip assembly 1909 to open the arm portion 1972—e.g., pivot the arm portion 1972 away from the attachment member 1986 in the direction of arrow 0—the threaded member 1974 can be rotated in a first direction via the component of the associated delivery system which engages the drive recess in the first head 1979a. Rotation of the threaded member 1974 in the first direction drives the actuation member 1975 upward toward the first horizontal portion 1976a along the threads of the rod 1980. As the actuation member 1975 moves upward, the projection 1985 moves along the slot 1983 from the first end portion 1984a toward the second end portion 1984b and drives the root portion 1971 to pivot about the pin 1982 in a clockwise direction and thus the arm portion 1972 to pivot open in the direction of the arrow 0. The clip assembly 1909 can reach a fully open position when the projection 1985 reaches the second end portion 1984b of the slot 1983. In some embodiments, the arm portion 1972 can pivot open by about 90° relative to the back member 1973 and the attachment member 1986. The second end portion 1984b can function as a hard stop that prevents further opening of the clip member 1970.

To actuate the clip assembly 1909 to close the arm portion 1972—e.g., pivot the arm portion 1972 toward the attachment member 1986 in the direction of arrow C—the threaded member 1974 can be rotated in a second direction opposite the first direction via the component of the associated delivery system which engages the drive recess in the first head 1979a. Rotation of the threaded member 1974 in the second direction drives the actuation member 1975 downward toward the second horizontal portion 1976b along the threads of the rod 1980. As the actuation member 1975 moves downward, the projection 1985 moves along the slot 1983 from the second end portion 1984b toward the first end portion 1984a and drives the root portion 1971 to pivot about the pin 1982 in a counterclockwise direction and thus the arm portion 1972 to pivot closed in the direction of the arrow C.

Some or all of the components of the clip assembly 1909 can be made from cobalt-chromium (Co-Cr), nitinol, stainless steel (e.g., SS316L stainless steel), and/or the like, and can be machined via, for example, wire electrical discharge machining (WEDM). In some embodiments, the attachment member 1986 can be formed from nitinol and/or another suitable material and can be electropolished. In some embodiments, the attachment member 1986 and the arm portion 1972 of the clip member 1970 are formed from nitinol, and the rest of the components of the clip assembly 1909 are formed from stainless steel. In some embodiments, the arm portion 1972 can be relatively flexible such as, for example, when the arm portion 1972 is formed of nitinol. In some aspects of the present technology, the flexible arm portion 1972 can be configured to sustain anatomical loads but to flex or deform in response to stronger-than-anatomical loads. This can enable recapture of an implantable device employing the clip assembly 1909. For example, if the clip assembly 1909 cannot be fully closed after deployment of the implantable device and before recapture of the implantable device, the implantable device can still be retracted into a catheter of an associated delivery system because the arm portion 1972 will flex (e.g., evert) as the implantable device is withdrawn into the catheter rather than catching against the end of the catheter and preventing recapture.

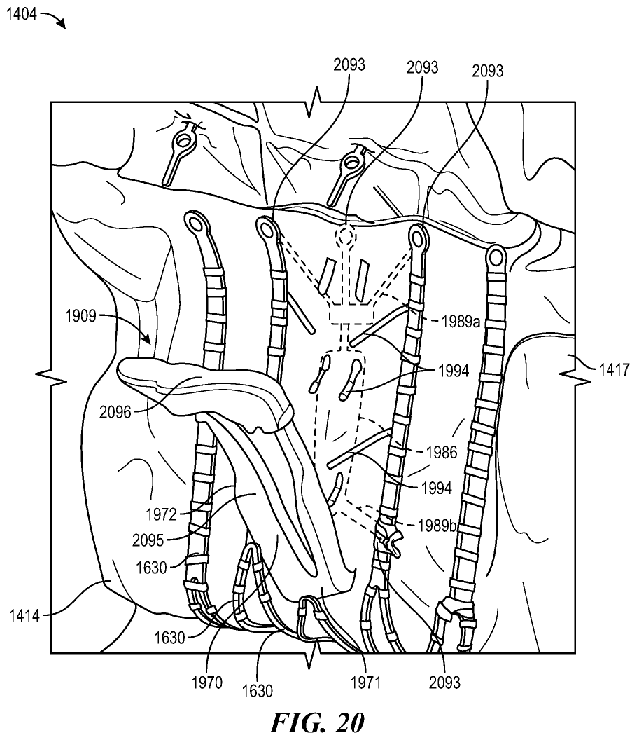

FIG. 20 is an enlarged perspective side view (e.g., an enlarged perspective posterior side view) of the coaptation member 1404 of FIGS. 14A and 14B including the clip assembly 1909 of FIGS. 19A and 19B in accordance with embodiments of the present technology. The clip assembly 1909 is shown in an open or partially open position in FIG. 20 in which the clip member 1970 is pivoted away from the posterior portion 1414 of the coaptation member 1404. Referring to FIGS. 19A-20, the attachment member 1986 (show in dashed line in FIG. 20) of the clip assembly 1909 is secured to the coaptation member 1404 via suturing 2093 that connects one or more of the first and second finger portions 1989a-b (e.g., the apertures 1990; obscured in FIG. 20) to one or more of the struts 1630 and the covering 1417 of the coaptation member 1404. The covering 1417 can cover the attachment member 1986. The coaptation member 1404 can include one or more cleats 1994 at the posterior portion 1414 in the region behind the clip member 1970 (e.g., between the attachment member 1986 the arm portion 1972) and projecting past the covering 1417. The cleats 1994 can be downward facing and configured to fix to a native leaflet clamped by the clip assembly 1909. In some aspects of the present technology, the cleats 1994 can improve fixation of the coaptation member 1404 to the native leaflet and inhibit atrial migration of the coaptation member 1404.

Referring to FIG. 20, the clip assembly 1909 further includes (i) a first fabric layer 2095 around the arm portion 1972 and at least a portion of the root portion 1971 of the clip member 1970 and (ii) a second fabric layer 2096 around at least a portion of the arm portion 1972 (e.g., the second segment 1992 thereof shown in FIGS. 19A and 19B). The first and second fabric layers 2095, 2096 can comprise a biocompatible material such as PTFE or ePTFE. In some embodiments, the first fabric layer 2095 comprises PTFE and the second fabric layer 2096 comprises ePTFE. In some embodiments, the second fabric layer 2096 is thicker than the first fabric layer 2095.

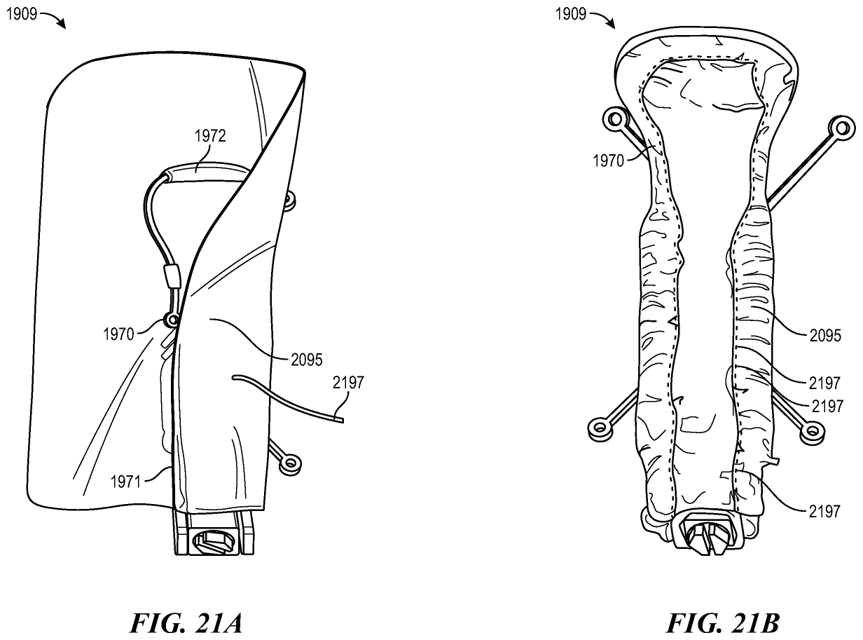

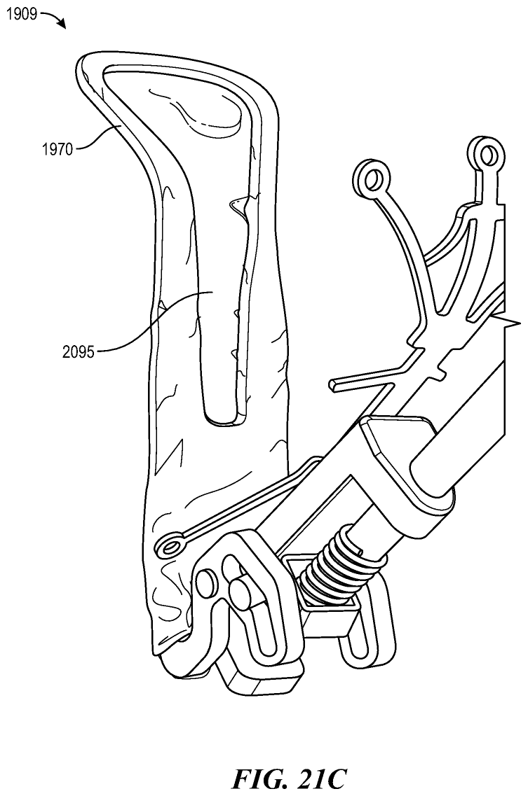

The first and second fabric layers 2095, 2096 can be secured to the clip member 1970 via suturing before the clip assembly 1909 is secured to coaptation member 1404. More specifically, for example, FIGS. 21A-21C are side views (e.g., a posterior side view, a posterior side view, and a perspective anterior side view, respectively) illustrating the sequential attachment of the first fabric layer 2095 to the clip member 1970 in accordance with embodiments of the present technology. Referring to FIG. 20A, the first fabric layer 2095 can be wrapped or folded around the arm portion 1972 and a portion of the root portion 1971 of the clip member 1970, and then a suture 2197 can be inserted through the folded first fabric layer 2095. Referring to FIGS. 21A and 21B, the suture 2197 can then be repeatedly inserted (e.g., using a needle coupled to the suture 2197) through the folded first fabric layer 2095 and wound about the arm portion 1972 and the root portion 1971 to secure the first fabric layer 2095 to the clip member 1970. FIG. 20C shows the clip assembly 1909 in an open or partially open configuration after the first fabric layer 2095 is secured to the clip member 1970.

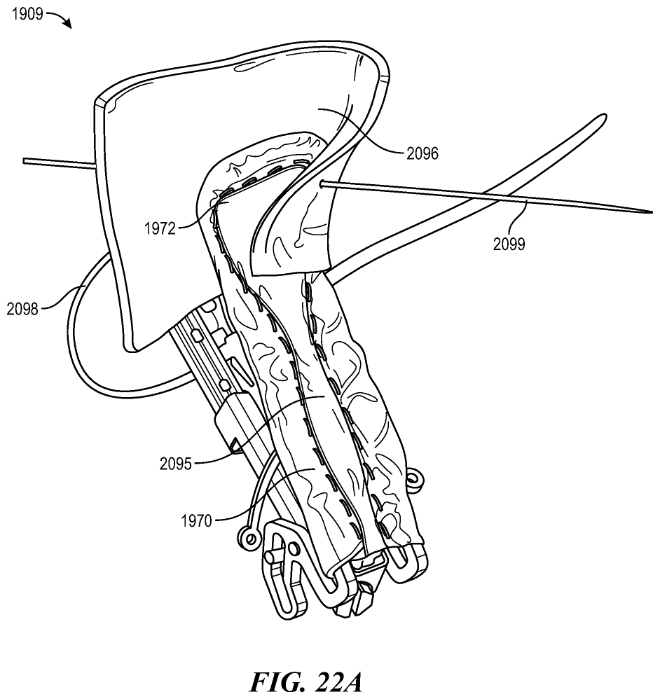

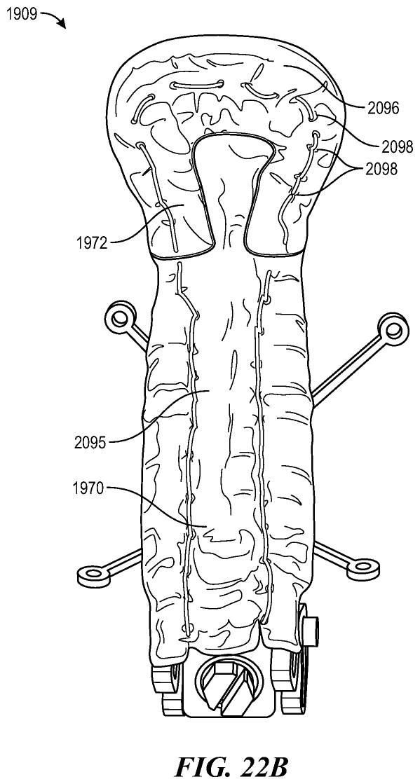

FIGS. 22A and 22B are a perspective side view (e.g., a perspective posterior side view) and a side view (e.g., a posterior side view) illustrating the sequential attachment of the second fabric layer 2096 to the clip member 1970 in accordance with embodiments of the present technology. Referring to FIG. 22A, the second fabric layer 2096 can be wrapped or folded around at least a portion of the arm portion 1972 (e.g., all or a portion of the second segment 1992 shown in FIGS. 19A and 19B) of the clip member 1970 and over the first fabric layer 2095 thereon. A suture 2197 can then be inserted through the folded second fabric layer 2096 via a needle 2099. Referring to Referring to FIGS. 22A and 22B, the suture 2198 can then be repeatedly inserted (e.g., using the needle 2099) through the folded second fabric layer 2096 and wound about the arm portion to secure the second fabric layer 2096 to the arm portion 1972.

The following examples are illustrative of several embodiments of the present technology:

1. A valve repair device for repairing a cardiac valve including a first native leaflet and a second native leaflet opposite the first native leaflet, the valve repair device comprising:
a coaptation member comprising (a) an inner portion having a coaptation surface configured to coapt with the first native leaflet during systole and (b) an outer portion configured to displace at least a portion of the second native leaflet; and
an atrial-fixation member comprising a plurality of interconnected struts having a circumferential U-like shape about a flow axis of the cardiac valve, wherein the atrial-fixation member extends upward from the coaptation member relative to the flow axis, wherein the interconnected struts define a brim portion and a pair of connection portions, wherein the connection portions are coupled to the coaptation member, and wherein the brim portion is configured to press against cardiac tissue above the first native leaflet proximate to a native valve annulus of the cardiac valve.

2. The valve repair device of example 1 wherein the atrial-fixation member is configured not to press against cardiac tissue above the second native leaflet proximate to the native valve annulus.

3. The valve repair device of example 1 or example 2 wherein the brim portion is positioned above the coaptation surface along the flow axis.

4. The valve repair device of any one of examples 1-3 wherein the atrial-fixation member has a medial portion between the connection portions and the brim portion, and wherein a superior portion of the atrial-fixation member tapers downward relative to the flow axis from the medial portion to the brim portion.

5. The valve repair device of any one of examples 1-4 wherein the brim portion includes a plurality of cleats extending upward relative to the flow axis.

6. The valve repair device of any one of examples 1-5 wherein the brim portion includes two or more connectors configured to be coupled to a delivery system for maintaining the coaptation member rotationally stable during implantation of the valve repair device at the cardiac valve.

7. The valve repair device of any one of examples 1-6 wherein the coaptation member includes a secondary brim portion configured to press against cardiac tissue above the second native leaflet proximate to the native valve annulus.

8. The valve repair device of example 7 wherein the secondary brim portion comprises a plurality of interconnected struts covered with a biocompatible material.

9. The valve repair device of example 7 or example 8 wherein the secondary brim portion curves in a direction away from the brim portion of the atrial-fixation member back toward the brim portion of the atrial-fixation member.

10. The valve repair device of any one of examples 7-9 wherein the coaptation member comprises a plurality of interconnected struts defining the secondary brim portion and a hollow interior volume within the coaptation member, and wherein the struts are covered with a biocompatible material.

11. The valve repair device of any one of examples 7-10, further comprising two or more cleats extending from the secondary brim portion downward relative to the flow axis.

12. The valve repair device of any one of examples 1-11 wherein the coaptation member includes (a) a first side portion between the inner portion and the outer portion and (b) a second side portion, opposite the first side portion, between the inner portion and the outer portion, wherein a first one of the connection portions is coupled proximate to the first side portion of the coaptation member, and wherein a second one of the connection portions is coupled proximate to the second side portion of the coaptation member.

13. The valve repair device of any one of examples 1-12, further comprising:
a clip assembly coupled to the coaptation member and configured to secure the second native leaflet, wherein the clip assembly comprises:
   a back member;
   a clip member having an arm portion and a root portion, wherein the root portion is pivotably coupled to the back member, and wherein the root portion defines a slot;
   a threaded member coupled to the back member and configured to rotate relative to the back member; and
   an actuation member coupled to the threaded member, wherein the actuation member includes a projection extending at least partially into the slot,
      wherein rotation of the threaded member in a first direction is configured to drive the actuation member in a first direction along the threaded member to drive the arm portion to pivot away from the outer portion of the coaptation member toward an open position, and
      wherein rotation of the threaded member in a second direction is configured to drive the actuation member in a second direction along the threaded member to drive the arm portion to pivot toward the outer portion of the coaptation member toward a closed position.

14. The valve repair device of example 13, further comprising an attachment member coupled to the back member between the back member and the arm portion, wherein the attachment member is secured to the coaptation member.

15. The valve repair device of example 14 wherein the attachment member is secured to the coaptation member via suturing.

16. The clip assembly of example 14 or example 15 wherein, when the arm portion is in the closed position—
the attachment member is separated from the arm portion by a gap, and
the gap has a generally uniform dimension along a length of the arm portion.

17. The valve repair device of any one of examples 13-16, further comprising a plurality of cleats extending downward from the coaptation member relative to the flow axis and toward the clip member.

18. A valve repair device for repairing a cardiac valve including a first native leaflet and a second native leaflet opposite the first native leaflet, the valve repair device comprising:
a coaptation member comprising (a) an inner portion having a coaptation surface configured to coapt with the first native leaflet during systole and (b) an outer portion configured to displace at least a portion of the second native leaflet;
an atrial-fixation member comprising a plurality of interconnected struts having a curved shape about a flow axis of the cardiac valve, wherein the atrial-fixation member is configured to press against cardiac tissue above the first native leaflet proximate to a native valve annulus of the cardiac valve; and
a pair of arm members extending between the coaptation member and the atrial-fixation member, wherein the arm members each have (a) a first portion that extends upward from the coaptation member relative to the flow axis and (b) a second portion extending from the first portion that curves downward toward the atrial-fixation member relative to the flow axis.

19. The valve repair device of example 18 wherein the arm members each comprise a nitinol strut.

20. The valve repair device of example 18 or example 19 wherein the arm members each comprise a pair of elongate first struts connected by a plurality of interconnected second struts having a diamond-like shape.

21. The valve repair device of any one of examples 18-20 wherein the atrial-fixation member is configured not to press against cardiac tissue above the second native leaflet proximate to the native valve annulus.

22. The valve repair device of any one of examples 18-21 wherein the brim portion is positioned above the coaptation surface along the flow axis.

23. The valve repair device of any one of examples 18-22 wherein the brim portion includes a plurality of cleats extending upward relative to the flow axis.

24. The valve repair device of any one of examples 18-23 wherein the coaptation member includes a brim portion configured to press against cardiac tissue above the second native leaflet proximate to the native valve annulus.

25. The valve repair device of any one of examples 18-24 wherein the coaptation member includes (a) a first side portion between the inner portion and the outer portion and (b) a second side portion, opposite the first side portion, between the inner portion and the outer portion, wherein the first portion of a first one of the arm members is coupled to the coaptation member proximate to the first side portion of the coaptation member, and wherein the first portion of a second one of the connection portions is coupled to the coaptation member proximate to the second side portion of the coaptation member.

26. The valve repair device of any one of examples 18-25, further comprising:
a clip assembly coupled to the coaptation member and configured to secure the second native leaflet, wherein the clip assembly comprises:
   a back member;
   a clip member having an arm portion and a root portion, wherein the root portion is pivotably coupled to the back member, and wherein the root portion defines a slot;
   a threaded member coupled to the back member and configured to rotate relative to the back member; and
   an actuation member coupled to the threaded member, wherein the actuation member includes a projection extending at least partially into the slot,
      wherein rotation of the threaded member in a first direction is configured to drive the actuation member in a first direction along the threaded member to drive the arm portion to pivot away from the outer portion of the coaptation member toward an open position, and
      wherein rotation of the threaded member in a second direction is configured to drive the actuation member in a second direction along the threaded member to drive the arm portion to pivot toward the outer portion of the coaptation member toward a closed position.

27. The valve repair device of example 26, further comprising an attachment member coupled to the back member between the back member and the arm portion, wherein the attachment member is secured to the coaptation member.

28. The valve repair device of example 27 wherein the attachment member is secured to the coaptation member via suturing.

29. The clip assembly of example 27 or example 28 wherein, when the arm portion is in the closed position—
the attachment member is separated from the arm portion by a gap, and
the gap has a generally uniform dimension along a length of the arm portion.

30. The valve repair device of any one of examples 26-29, further comprising a plurality of cleats extending downward from the coaptation member relative to the flow axis and toward the clip member.

31. A clip assembly for securing a native leaflet of a cardiac valve, the clip assembly comprising:
a back member;
a clip member having an arm portion and a root portion, wherein the root portion is pivotably coupled to the back member, and wherein the root portion defines a slot;
a threaded member coupled to the back member and configured to rotate relative to the back member; and
an actuation member coupled to the threaded member, wherein the actuation member includes a projection extending at least partially into the slot,
   wherein rotation of the threaded member in a first direction is configured to drive the actuation member in a first direction along the threaded member to drive the arm portion to pivot away from the back member toward an open position, and
   wherein rotation of the threaded member in a second direction is configured to drive the actuation member in a second direction along the threaded member to drive the arm portion to pivot toward the back member toward a closed position.

32. The clip assembly of example 31 wherein the actuation member is a nut.

33. The clip assembly of example 31 or example 32, further comprising an attachment member coupled to the back member between the back member and the arm portion.

34. The clip assembly of example 33 wherein the attachment member includes a body portion and a plurality of finger portions extending from the body portion, and wherein two or more of the finger portions are configured to be secured to a coaptation member configured to displace at least a portion of the native leaflet.

35. The clip assembly of example 33 or example 34 wherein the attachment member has a planform shape that generally matches a planform shape of the arm portion.

36. The clip assembly of any one of examples 33-35 wherein the arm portion has a first flat segment and a first curved segment, wherein the attachment member has a second flat segment and a second curved segment, and wherein, when the arm portion is in the closed position, (a) the first flat segment is positioned adjacent to and generally parallel to the second flat segment and (b) the first curved segment is positioned adjacent to and generally parallel to the second curved segment.

37. The clip assembly of any one of examples 33-36 wherein, when the arm portion is in the closed position—
the attachment member is separated from the arm portion by a gap, and
the gap has a generally uniform dimension along a length of the arm portion.

38. A valve repair device for repairing a cardiac valve, the valve repair device comprising:
a coaptation member comprising (a) an inner portion having a coaptation surface configured to coapt with a first native leaflet during systole, (b) an outer portion configured to displace at least a portion of a second native leaflet opposing the first native leaflet, and (c) a brim portion configured to engage cardiac tissue only in a region superior to the second native leaflet proximate to the native valve annulus; and
an atrial-fixation member coupled to coaptation member, wherein the atrial-fixation member is configured to engage cardiac tissue only in a region superior to the first native leaflet, and wherein the atrial-fixation member and the brim portion cooperate to maintain the coaptation member in a substantially stationary position during cardiac cycles.

39. The valve repair device of example 38 wherein the atrial-fixation member is configured not to press against cardiac tissue superior to the second native leaflet.

40. The valve repair device of example 38 or example 39 wherein the brim portion comprises a plurality of interconnected struts covered with a biocompatible material.

41. The valve repair device of any one of examples 38-40 wherein the brim portion has a shape that curves in a direction away from the atrial-fixation member back toward the atrial-fixation member.

42. The valve repair device of any one of examples 38-41 wherein the coaptation member comprises a plurality of interconnected struts defining the brim portion and a hollow interior volume within the coaptation member, and wherein the struts are covered with a biocompatible material.

43. The valve repair device of any one of examples 38-42, further comprising two or more cleats extending from the brim portion downward relative to a flow axis of the cardiac valve and configured to engage the cardiac tissue superior to the second native leaflet proximate to the native valve annulus.

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments can perform steps in a different order. The various embodiments described herein can also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms can also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications can be made without deviating from the technology. Further, while advantages associated with some embodiments of the technology have been described in the context of those embodiments, other embodiments can also exhibit such advantages, and not all embodiments need necessarily exhibit such advantages to fall within the scope of the technology. Accordingly, the disclosure and associated technology can encompass other embodiments not expressly shown or described herein.

I/We claim:

1. A valve repair device for repairing a cardiac valve including a first native leaflet and a second native leaflet opposite the first native leaflet, the valve repair device comprising:
a coaptation member comprising (a) an inner portion having a coaptation surface configured to coapt with the first native leaflet during systole and (b) an outer portion configured to displace at least a portion of the second native leaflet; and
an atrial-fixation member comprising a plurality of interconnected struts having a circumferential U-like shape about a flow axis of the cardiac valve, wherein the atrial-fixation member extends upward from the coaptation member relative to the flow axis, wherein the interconnected struts define a brim portion and a pair of connection portions, wherein the connection portions are coupled to the coaptation member, and wherein the brim portion is configured to press against cardiac tissue above the first native leaflet proximate to a native valve annulus of the cardiac valve.

2. The valve repair device of claim 1 wherein the atrial-fixation member is configured not to press against cardiac tissue above the second native leaflet proximate to the native valve annulus.

3. The valve repair device of claim 1 wherein the brim portion is positioned above the coaptation surface along the flow axis.

4. The valve repair device of claim 1 wherein the atrial-fixation member has a medial portion between the connection portions and the brim portion, and wherein a superior portion of the atrial-fixation member tapers downward relative to the flow axis from the medial portion to the brim portion.

5. The valve repair device of claim 1 wherein the brim portion includes a plurality of cleats extending upward relative to the flow axis.

6. The valve repair device of claim 1 wherein the brim portion includes two or more connectors configured to be coupled to a delivery system for maintaining the coaptation member rotationally stable during implantation of the valve repair device at the cardiac valve.

7. The valve repair device of claim 1 wherein the coaptation member includes a secondary brim portion configured to press against cardiac tissue above the second native leaflet proximate to the native valve annulus.

8. The valve repair device of claim 7 wherein the secondary brim portion comprises a plurality of interconnected struts covered with a biocompatible material.

9. The valve repair device of claim 7 wherein the secondary brim portion curves in a direction away from the brim portion of the atrial-fixation member back toward the brim portion of the atrial-fixation member.

10. The valve repair device of claim 7 wherein the coaptation member comprises a plurality of interconnected struts defining the secondary brim portion and a hollow interior volume within the coaptation member, and wherein the struts are covered with a biocompatible material.

11. The valve repair device of claim 7, further comprising two or more cleats extending from the secondary brim portion downward relative to the flow axis.

12. The valve repair device of claim 1 wherein the coaptation member includes (a) a first side portion between the inner portion and the outer portion and (b) a second side portion, opposite the first side portion, between the inner portion and the outer portion, wherein a first one of the connection portions is coupled proximate to the first side portion of the coaptation member, and wherein a second one of the connection portions is coupled proximate to the second side portion of the coaptation member.

13. The valve repair device of claim 1, further comprising:
a clip assembly coupled to the coaptation member and configured to secure the second native leaflet, wherein the clip assembly comprises:
a back member;
a clip member having an arm portion and a root portion, wherein the root portion is pivotably coupled to the back member, and wherein the root portion defines a slot;
a threaded member coupled to the back member and configured to rotate relative to the back member; and
an actuation member coupled to the threaded member, wherein the actuation member includes a projection extending at least partially into the slot,
wherein rotation of the threaded member in a first direction is configured to drive the actuation member in a first direction along the threaded member to drive the arm portion to pivot away from the outer portion of the coaptation member toward an open position, and
wherein rotation of the threaded member in a second direction is configured to drive the actuation member in a second direction along the threaded member to drive the arm portion to pivot toward the outer portion of the coaptation member toward a closed position.

14. The valve repair device of claim 13, further comprising an attachment member coupled to the back member between the back member and the arm portion, wherein the attachment member is secured to the coaptation member.

15. The valve repair device of claim 14 wherein the attachment member is secured to the coaptation member via suturing.

16. The clip assembly of claim 14 wherein, when the arm portion is in the closed position—
the attachment member is separated from the arm portion by a gap, and
the gap has a generally uniform dimension along a length of the arm portion.

17. The valve repair device of claim 13, further comprising a plurality of cleats extending downward from the coaptation member relative to the flow axis and toward the clip member.

18. A valve repair device for repairing a cardiac valve including a first native leaflet and a second native leaflet opposite the first native leaflet, the valve repair device comprising:
a coaptation member comprising (a) an inner portion having a coaptation surface configured to coapt with the first native leaflet during systole and (b) an outer portion configured to displace at least a portion of the second native leaflet;
an atrial-fixation member comprising a plurality of interconnected struts having a curved shape about a flow axis of the cardiac valve, wherein the atrial-fixation member is configured to press against cardiac tissue above the first native leaflet proximate to a native valve annulus of the cardiac valve; and
a pair of arm members extending between the coaptation member and the atrial-fixation member, wherein the arm members each have (a) a first portion that extends upward from the coaptation member relative to the flow axis and (b) a second portion extending from the first portion that curves downward toward the atrial-fixation member relative to the flow axis.

19. The valve repair device of claim 18 wherein the arm members each comprise a nitinol strut.

20. The valve repair device of claim 18 wherein the arm members each comprise a pair of elongate first struts connected by a plurality of interconnected second struts having a diamond-like shape.

21. The valve repair device of claim 18 wherein the atrial-fixation member is configured not to press against cardiac tissue above the second native leaflet proximate to the native valve annulus.

22. The valve repair device of claim 18 wherein the brim portion is positioned above the coaptation surface along the flow axis.

23. The valve repair device of claim 18 wherein the brim portion includes a plurality of cleats extending upward relative to the flow axis.

24. The valve repair device of claim 18 wherein the coaptation member includes a brim portion configured to press against cardiac tissue above the second native leaflet proximate to the native valve annulus.

25. The valve repair device of claim 18 wherein the coaptation member includes (a) a first side portion between the inner portion and the outer portion and (b) a second side portion, opposite the first side portion, between the inner portion and the outer portion, wherein the first portion of a first one of the arm members is coupled to the coaptation member proximate to the first side portion of the coaptation member, and wherein the first portion of a second one of the connection portions is coupled to the coaptation member proximate to the second side portion of the coaptation member.

26. The valve repair device of claim 18, further comprising:
a clip assembly coupled to the coaptation member and configured to secure the second native leaflet, wherein the clip assembly comprises:
a back member;
a clip member having an arm portion and a root portion, wherein the root portion is pivotably coupled to the back member, and wherein the root portion defines a slot;
a threaded member coupled to the back member and configured to rotate relative to the back member; and
an actuation member coupled to the threaded member, wherein the actuation member includes a projection extending at least partially into the slot,
wherein rotation of the threaded member in a first direction is configured to drive the actuation member in a first direction along the threaded member to drive the arm portion to pivot away from the outer portion of the coaptation member toward an open position, and
wherein rotation of the threaded member in a second direction is configured to drive the actuation member in a second direction along the threaded member to drive the arm portion to pivot toward the outer portion of the coaptation member toward a closed position.

27. The valve repair device of claim 26, further comprising an attachment member coupled to the back member between the back member and the arm portion, wherein the attachment member is secured to the coaptation member.

28. The valve repair device of claim 27 wherein the attachment member is secured to the coaptation member via suturing.

29. The clip assembly of claim 27 wherein, when the arm portion is in the closed position—
the attachment member is separated from the arm portion by a gap, and
the gap has a generally uniform dimension along a length of the arm portion.

30. The valve repair device of claim 26, further comprising a plurality of cleats extending downward from the coaptation member relative to the flow axis and toward the clip member.

31. A clip assembly for securing a native leaflet of a cardiac valve, the clip assembly comprising:
a back member;
a clip member having an arm portion and a root portion, wherein the root portion is pivotably coupled to the back member, and wherein the root portion defines a slot;
a threaded member coupled to the back member and configured to rotate relative to the back member; and
an actuation member coupled to the threaded member, wherein the actuation member includes a projection extending at least partially into the slot,
wherein rotation of the threaded member in a first direction is configured to drive the actuation member in a first direction along the threaded member to drive the arm portion to pivot away from the back member toward an open position, and
wherein rotation of the threaded member in a second direction is configured to drive the actuation member in a second direction along the threaded member to drive the arm portion to pivot toward the back member toward a closed position.

32. The clip assembly of claim 31 wherein the actuation member is a nut.

33. The clip assembly of claim 31, further comprising an attachment member coupled to the back member between the back member and the arm portion.

34. The clip assembly of claim 33 wherein the attachment member includes a body portion and a plurality of finger portions extending from the body portion, and wherein two or more of the finger portions are configured to be secured to a coaptation member configured to displace at least a portion of the native leaflet.

35. The clip assembly of claim 33 wherein the attachment member has a planform shape that generally matches a planform shape of the arm portion.

36. The clip assembly of claim 33 wherein the arm portion has a first flat segment and a first curved segment, wherein the attachment member has a second flat segment and a second curved segment, and wherein, when the arm portion is in the closed position, (a) the first flat segment is positioned adjacent to and generally parallel to the second flat segment and (b) the first curved segment is positioned adjacent to and generally parallel to the second curved segment.

37. The clip assembly of claim 33 wherein, when the arm portion is in the closed position—
the attachment member is separated from the arm portion by a gap, and
the gap has a generally uniform dimension along a length of the arm portion.

38. A valve repair device for repairing a cardiac valve, the valve repair device comprising:
a coaptation member comprising (a) an inner portion having a coaptation surface configured to coapt with a first native leaflet during systole, (b) an outer portion configured to displace at least a portion of a second native leaflet opposing the first native leaflet, and (c) a brim portion configured to engage cardiac tissue only in a region superior to the second native leaflet proximate to the native valve annulus; and
an atrial-fixation member coupled to coaptation member, wherein the atrial-fixation member is configured to engage cardiac tissue only in a region superior to the first native leaflet, and wherein the atrial-fixation member and the brim portion cooperate to maintain the coaptation member in a substantially stationary position during cardiac cycles.

39. The valve repair device of claim 38 wherein the atrial-fixation member is configured not to press against cardiac tissue superior to the second native leaflet.

40. The valve repair device of claim 38 wherein the brim portion comprises a plurality of interconnected struts covered with a biocompatible material.

41. The valve repair device of claim 38 wherein the brim portion has a shape that curves in a direction away from the atrial-fixation member back toward the atrial-fixation member.

42. The valve repair device of claim 38 wherein the coaptation member comprises a plurality of interconnected struts defining the brim portion and a hollow interior volume within the coaptation member, and wherein the struts are covered with a biocompatible material.

43. The valve repair device of claim 38, further comprising two or more cleats extending from the brim portion downward relative to a flow axis of the cardiac valve and configured to engage the cardiac tissue superior to the second native leaflet proximate to the native valve annulus.
